SEQUENCE LISTING (19 sequences)

SEQ ID NO: 1 - protein, 1594 aa, Homo sapiens
MPCAQRSWLANLSVVAQLLNFGALCYGRQPQPGPVRFPDRRQEHFIKGLPEYHVVGPVRVDASGHFLSYGLHYPITSSRRKRDLDGSEDWVYYRISHEEKDLFFNLTVNQGFLSNSYIMEKRYGNLSHVKMMASSAPLCHLSGTVLQQGTRVGTAALSACHGLTGFFQLPHGDFFIEPVKKHPLVEGGYHPHIVYRRQKVPETKEPTCGLKDSVNISQKQELWREKWERHNLPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGWGRWSPWSHCSRTCGAGVQSAERLCNNPEPKFGGKYCTGERKRYRLCNVHPCRSEAPTFRQMQCSEFDTVPYKNELYHWFPIFNPAHPCELYCRPIDGQFSEKMLDAVIDGTPCFEGGNSRNVCINGICKMVGCDYEIDSNATEDRCGVCLGDGSSCQTVRKMFKQKEGSGYVDIGLIPKGARDIRVMEIEGAGNFLAIRSEDPEKYYLNGGFIIQWNGNYKLAGTVFQYDRKGDLEKLMATGPTNESVWIQLLFQVTNPGIKYEYTIQKDGLDNDVEQQMYFWQYGHWTECSVTCGTGIRRQTAHCIKKGRGMVKATFCDPETQPNGRQKKCHEKACPPRWWAGEWEACSATCGPHGEKKRTVLCIQTMVSDEQALPPTDCQHLLKPKTLLSCNRDILCPSDWTVGNWSECSVSCGGGVRIRSVTCAKNHDEPCDVTRKPNSRALCGLQQCPSSRRVLKPNKGTISNGKNPPTLKPVPPPTSRPRMLTTPTGPESMSTSTPAISSPSPTTASKEGDLGGKQWQDSSTQPELSSRYLISTGSTSQPILTSQSLSIQPSEENVSSSDTGPTSEGGLVATTTSGSGLSSSRNPITWPVTPFYNTLTKGPEMEIHSGSGEEREQPEDKDESNPVIWTKIRVPGNDAPVESTEMPLAPPLTPDLSRESWWPPFSTVMEGLLPSQRPTTSETGTPRVEGMVTEKPANTLLPLGGDHQPEPSGKTANRNHLKLPNNMNQTKSSEPVLTEEDATSLITEGFLLNASNYKQLTNGHGSAHWIVGNWSECSTTCGLGAYWRRVECSTQMDSDCAAIQRPDPAKRCHLRPCAGWKVGNWSKCSRNCSGGFKIREIQCVDSRDHRNLRPFHCQFLAGIPPPLSMSCNPEPCEAWQVEPWSQCSRSCGGGVQERGVFCPGGLCDWTKRPTSTMSCNEHLCCHWATGNWDLCSTSCGGGFQKRTVQCVPSEGNKTEDQDQCLCDHKPRPPEFKKCNQQACKKSADLLCTKDKLSASFCQTLKAMKKCSVPTVRAECCFSCPQTHITHTQRQRRQRLLQKSKEL

SEQ ID NO: 2 - DNA, 19 bp, synthetic construct
  source 1..19: mol_type = other DNA
aagtggtgatgggcttccc

SEQ ID NO: 3 - DNA, 20 bp, synthetic construct
  source 1..20: mol_type = other DNA
ggcaaattcaacggcacagt

SEQ ID NO: 4 - DNA, 18 bp, synthetic construct
  source 1..18: mol_type = other DNA
gtgtggaacaggtatccg

SEQ ID NO: 5 - DNA, 18 bp, synthetic construct
  source 1..18: mol_type = other DNA
cagagctcgactgttggg

SEQ ID NO: 6 - DNA, 20 bp, synthetic construct
  source 1..20: mol_type = other DNA
tgactggaagagcggagagt

SEQ ID NO: 7 - DNA, 15 bp, synthetic construct
  source 1..15: mol_type = other DNA
gttcgggctgatgta

SEQ ID NO: 8 - DNA, 15 bp, synthetic construct
  source 1..15: mol_type = other DNA
atctggacccctcct

SEQ ID NO: 9 - DNA, 15 bp, synthetic construct
  source 1..15: mol_type = other DNA
gcccagtgatttcag

SEQ ID NO: 10 - DNA, 20 bp, synthetic construct
  source 1..20: mol_type = other DNA
gaaggtgaaggtcggagtca

SEQ ID NO: 11 - DNA, 20 bp, synthetic construct
  source 1..20: mol_type = other DNA
tggactccacgacgtactca

SEQ ID NO: 12 - DNA, 18 bp, synthetic construct
  source 1..18: mol_type = other DNA
gtgtggaacaggtatccg

SEQ ID NO: 13 - DNA, 18 bp, synthetic construct
  source 1..18: mol_type = other DNA
cagagctcgactgttggg

SEQ ID NO: 14 - DNA, 21 bp, synthetic construct
  source 1..21: mol_type = other DNA
cccagccacaaagagtctaca

SEQ ID NO: 15 - DNA, 21 bp, synthetic construct
  source 1..21: mol_type = other DNA
attggtgggatgtcttcgtct

SEQ ID NO: 16 - RNA, 25 nt, synthetic construct
  source 1..25: mol_type = other RNA
caccgaacatcatagatcactccgg

SEQ ID NO: 17 - RNA, 25 nt, synthetic construct
  source 1..25: mol_type = other RNA
aaacccggagtgatctatgatgttc

SEQ ID NO: 18 - DNA, 27 bp, synthetic construct
  source 1..27: mol_type = other DNA
cacaattgcccaagcgctaggacacag

SEQ ID NO: 19 - DNA, 18 bp, synthetic construct
  source 1..18: mol_type = other DNA
aaagccagagggagtccc

(19) United States
(12) Patent Application Publication
(10) Pub. No.: US 2026/0202408 A1
(43) Pub. Date: Jul. 16, 2026

(54) ADAMTS12 AS A TARGET MOLECULE FOR THE TREATMENT OF CHRONIC RENAL INSUFFICIENCY AND RENAL FIBROSIS

(71) Applicant: RHEINISCH-WESTFÄLISCHE TECHNISCHE HOCHSCHULE (RWTH) AACHEN, Aachen (DE)

(72) Inventors: Rafael KRAMANN, Lemiers (NL); Konrad HOEFT, Aachen (DE); Lars Leonard KOCH, Aachen (DE)

(73) Assignee: RHEINISCH-WESTFÄLISCHE TECHNISCHE HOCHSCHULE (RWTH) AACHEN, Aachen (DE)

(21) Appl. No.: 19/134,899

(22) PCT Filed: Dec. 5, 2023
(86) PCT No.: PCT/EP2023/084256

§ 371 Date: Jun. 2, 2025

(30) Foreign Application Priority Data
Dec. 5, 2022 (DE) 10 2022 132 156.8

(51) Int. Cl. G01N 33/573; C12N 9/22; C12N 15/11

(52) U.S. Cl. CPC G01N 33/573; C12N 9/226; C12N 15/111; C12N 2310/20; C12N 2500/10; G01N 2333/96419

(57) ABSTRACT

The present invention relates to the role of ADAMTS12 protein (A Disintegrin And Metalloproteinase with ThromboSpondin motifs 12 protein) in the development of chronic kidney disease, in particular progressive chronic kidney disease and renal fibrosis. In particular, the present invention relates to methods for identifying compounds that bind to the ADAMTS12 protein and to the use of ADAMTS12 protein for screening and identifying ADAMTS12-interacting and ADAMTS12-inhibiting compounds. The present invention further relates to pharmaceutical compositions for use in the treatment of kidney diseases, in particular pharmaceutical compositions comprising active agents that bind to and/or inhibit the ADAMTS12 protein.

This application is a national phase of International Application No. PCT/EP2023/084256 filed Dec. 5, 2023, which claims priority to German Application No. 10 2022 132 156.8 filed Dec. 5, 2022, each of which is hereby incorporated herein by reference in its entirety.

### INCORPORATION BY REFERENCE

The material in the Sequence Listing XML file named 'MH RD42278—Sequence Listing ST 26.xml', created on Jul. 2, 2025, and having a size of 19,512 bytes, is incorporated herein by reference in its entirety.

### TECHNICAL FIELD OF THE INVENTION

The present invention relates to the role of ADAMTS12 protein (A Disintegrin And Metalloproteinase with ThromboSpondin motifs 12 protein) in the development of chronic kidney and heart disease, in particular progressive chronic renal insufficiency and renal fibrosis and heart failure and cardiac fibrosis, respectively. In particular, the present invention relates to methods for identifying compounds that bind to and/or inhibit the ADAMTS12 protein and the use of ADAMTS12 protein for screening and identifying ADAMTS12-interacting and ADAMTS12-inhibiting compounds. The present invention further relates to pharmaceutical compositions for use in the treatment of kidney diseases, in particular pharmaceutical compositions comprising active agents that bind to and/or inhibit the ADAMTS12 protein.

### BACKGROUND AND PRIOR ART

Fibrosis is defined as the pathological deposition of extracellular connective tissue (extracellular matrix, ECM), which is associated with a displacement of healthy tissue and a loss of organ function. While the initial deposition of ECM is important to maintain tissue integrity after organ damage, the uncontrolled deposition of ECM leads to the displacement of healthy tissue and a loss of organ function. Irrespective of the initial damage, fibrosis is the common end stage of almost all chronic diseases across all organs. Current estimates assume that fibrosis is therefore responsible for up to 45% of all deaths in industrialised countries (Henderson et al., 2020). Due to the ageing population, the prevalence of fibrosis will continue to increase in the coming decades.

The number of patients suffering from chronic renal insufficiency (chronic kidney disease, or CKD) is increasing worldwide, and current data shows that up to 10% of the population in Western countries will develop CKD in the course of their lives (Jha et al., 2013). Due to the rising average age and the increasing prevalence of hypertension and diabetes, it can be assumed that the incidence of CKD will continue to rise in the future. As kidney function declines, morbidity and mortality will increase significantly. In the final stage of CKD, dialysis and kidney transplantation are the only treatment options. Due to the long waiting times for donated kidneys, most of these patients are dialysed. However, dialysis therapy is associated with a high mortality rate (5-year survival after new dialysis requirement approx. 50%, Naylor et al., 2019), numerous co-morbidities and a significant reduction in quality of life (4-6 hours of dialysis therapy, 3 times a week). In addition, the high costs of dialysis represent an enormous economic burden for the healthcare system (Cm and F, 2017). Novel therapeutic approaches are therefore required.

The extent of renal fibrosis is inextricably linked to the loss of renal function and the clinical course of CKD. Renal fibrosis is characterised by high expression, secretion and accumulation of extracellular matrix (ECM) proteins such as collagen-1.

Myofibroblasts, which expand after organ damage, are the main producers of the extracellular matrix and thus play a key role in the development of fibrosis (Henderson et al, 2020; Kuppe et al., 2021). While the origin of these myofibroblasts was unclear for a long time, a perivascular cell population has now been identified that is characterised by the expression of the transcription factor Gli1 and from which 50% of myofibroblasts originate (Kramann et al., 2015a). However, it is still unclear which signals lead to activation, expansion and myofibroblast differentiation of these Gli1 fibroblasts.

The histological structure of the kidney can be divided into three main compartments, all of which can be affected by fibrosis, specifically referred to as glomerulosclerosis in the glomeruli, interstitial fibrosis in the tubulointerstitium, and arteriosclerosis and perivascular fibrosis in the vasculature (Djudjai and Boor 2019).

In animal models, the inhibition of fibrosis can prevent the progression of chronic renal insufficiency and preserve kidney function (Kramann et al., 2015a, 2015b). However, there is currently no approved therapy for renal fibrosis. Due to the increasing prevalence of chronic renal insufficiency, the development of drugs to treat fibrosis is therefore essential.

Thus, an underlying object of the present invention is to provide methods and means for identifying active agents, compounds and compositions, and said active agents, compounds and compositions for use in the treatment of chronic kidney disease.

The present application discloses the identification of a new molecular target for the therapy of renal fibrosis. Based on an identification and isolation of Gli1-expressing fibroblasts after induction of renal fibrosis from a mouse, and an analysis of the total RNA, i.e. the expression of all genes expressed by said activated fibroblasts, by means of a microarray, the inventors were surprisingly able to identify the protein “A Disintegrin And Metalloproteinase with ThromboSpondin motifs 12” (ADAMTS12) as a new molecular target. ADAMTS12 belongs to the family of ADAMTS metalloproteases and degrades the extracellular matrix protein thrombospondin 5 (Wei et al., 2014). The inventors were able to show for the first time that the metalloprotease ADAMTS12 is an essential mediator of fibrosis and that the knockout (KO) of ADAMTS12 inhibits the development of fibrosis after kidney and heart damage.

The ADAMTS (“A Disintegrin And Metalloproteinase with ThromboSpondin motifs”) proteins belong to the metzincin protease superfamily, which are named after a conserved methionine residue near the active centre of the Zn-ion-dependent metalloproteinases (Kelwick et al. 2015). At least 19 different ADAMTS proteins have been identified in mammalian genomes to date. The ADAMTS proteins are secreted, extracellular zinc-matrix metalloproteinases with a uniform, ordered modular structure. The ADAMTS proteins are initially expressed as inactive pre-proenzymes whose structures contain a signal peptide, a variable length pro-region, a catalytic metalloproteinase domain, a disintegrin-like domain, a central thrombospondin type 1-like (TSP) sequence repeat, a cysteine-rich domain, a spacer region, and a variable number of additional C-terminal TSP repeats (FIG. 5) (Kelwick et al. 2015; Lin et al. 2009).

The ADAMTS12 gene contains a total of 24 exons that encode an extracellular protein of 1594 amino acids (Mohamedi et al. 2021). Aggrecan, COMP (cartilage oligomeric matrix protein) and alpha2M (alpha 2-macroglobulin) have been identified as substrates of ADAMTS12. A role of ADAMTS12 protein has been described in chondrogenesis, cartilage development and gonadal differentiation, as well as in paediatric stroke, schizophrenia, tumourigenesis and arthritis (Lin et al. 2009; Wei et al. 2014; Mohamedi et al. 2021; Witten et al. 2020).

The present application discloses ADAMTS12 as a target molecule and thus a new therapeutic approach for the development of therapeutics for the treatment of patients with chronic renal insufficiency and renal fibrosis.

### SUMMARY OF THE INVENTION

The present invention provides methods and means for identifying active agents, compounds and compositions for use in the treatment of chronic renal insufficiency, in particular for identifying highly effective active agents, compounds and compositions for use in the treatment of progressive chronic kidney disease and renal fibrosis.

In view of the prior art, it was therefore an object of the present invention to provide a method for reducing the expression and/or secretion of extracellular matrix (ECM) proteins by a particular cell. A further object of the present invention was to provide a method for reducing the expression, differentiation and secretion of extracellular matrix proteins by (myo)fibroblasts.

A further object of the present invention was to provide a method for identifying an active agent that binds to and/or inhibits the ADAMTS12 protein or a fragment thereof.

It was a further object of the present invention to provide a method for utilising a nucleic acid encoding the ADAMTS12 protein, or a fragment thereof, or the ADAMTS12 protein itself, or a fragment thereof, for the identification of an active agent which binds to ADAMTS12, or a fragment thereof.

It was a further object of the present invention to provide active agents for use in the treatment of chronic kidney disease, in particular for use in the treatment of progressive chronic kidney disease and/or renal fibrosis, based on the findings described above.

A further object of the present invention is to provide pharmaceutical compositions containing these agents and methods for the preparation of such pharmaceutical compositions, based on the above-described findings.

The described and further technical problems are solved by the devices or methods according to the independent claims of the current invention. The dependent claims describe preferred embodiments. Value ranges which are limited by numerical values should always include said limit values.

The invention and general advantageous embodiments are explained in greater detail below.

### DESCRIPTION OF THE DRAWINGS

FIG. 1: Microarray of Gil fibroblasts after unilateral ureteral obstruction (UUO). (A) Setup of the experiment. (B) Hallmark Gene Set Enrichment Analysis (GSEA) based on the differentially expressed genes in Gli1 fibroblasts after UUO. (C) Representation of the “Top 25” upregulated genes in Gli1 fibroblasts according to UUO ordered by T-values. (D) Representative images of in situ hybridisation (ISH) for Pdgfrb and Adamts12 transcripts in murine kidneys at different time points after ischaemia-reperfusion (IRI) (E) Quantification of Adamts12 ISH expression. (F) Quantification of Pdgfrb ISH expression. (G) Quantification of Adamts12 ISH expression in Pdgfrb positive cells. **p<0.01, ***p<0.00.

FIG. 2: Genetic loss of Adamts12 protects against fibrosis. (A-G) Adamts12^{-/-} or WT mice underwent unilateral ureteral obstruction (UUO) or placebo surgery (Sham). 10 days after surgery, the mice were killed and the kidneys were removed. (A) Adamts12 RT-qPCR. (B) Collagen 1 RT-qPCR (Col 1a1). (C) Fibronectin (Fn1) RT-qPCR. (D) PDGFRb immunofluorescence staining (IF). (E) Quantification of IF PDGFRb expression. (F) Immunohistochemical (IHC) staining of collagen 1 (Col 1). (G) Quantification of IHC collagen 1 expression. (H-I) Adamts12^{-/-} or WT mice underwent myocardial infarction (MI) or placebo surgery (Sham). (H) Echocardiographically measured left ventricular ejection fraction (LV-EF) in WT and Adamts12^{-/-} mice after myocardial infarction (MI) or sham surgery. (I) Fibrosis measured in serial sections of picrosirius red staining, in WT and Adamts12^{-/-} mice after myocardial infarction or sham surgery. *p<0.05, **p<0.01, ***p<0.001, ****p<0.001.

FIG. 3: ADAMTS12 CRISPR-Cas9 KO in human renal PDGFRb-positive fibroblasts. (A-B) ADAMTS12 or COL1A1 RT-qPCR in control (non-targeting gRNA) and ADAMTS12 CRISPR-KO (ADAMTS12-KO) human renal PDGFRb fibroblasts after stimulation with vehicle or TGFb. (C) Migration analysis of control (non-targeting gRNA) and ADAMTS12 CRISPR-KO (ADAMTS12-KO) human renal PDGFRb fibroblasts after stimulation with vehicle or TGFb. (D) Western blot for the HA epitope, tubulin, and eGFP in human renal PDGFRb fibroblasts with ADAMTS12 CRISPR-KO (KO) and empty expression plasmid pMIG (without insertion of a protein-coding nucleotide sequence), ADAMTS12 CRISPR-KO and overexpression of HA-“tagged” ADAMTS12 using pMIG expression plasmid (WT), and overexpression of catalytically inactive, HA-“tagged” ADAMTS12 protein using pMIG expression plasmid (Mut). (E) Migration analysis of human renal PDGFRb fibroblasts with ADAMTS12 CRISPR-KO (ADAMTS12-KO) and empty expression plasmid pMIG (without insertion of a protein-coding nucleotide sequence), ADAMTS12 CRISPR-KO and overexpression of the HA-“tagged” ADAMTS12 protein (ADAMTS12-KO with WT), and overexpression of the catalytically-inactive, HA-“tagged” ADAMTS12 protein (Mut).

FIG. 4: ADAMTS12 expression in human kidneys, (A) ADAMTS1 2 expression in CD10-negative, interstitium-enriched renal single cells (after depletion of proximal tubule cells) isolated from 15 human kidneys by FACS. (B) ADAMTS12 expression in PDGFRb-positive single cells isolated from eight human kidneys by FACS. (C) Representative image of ISH for PDGFRB, COL1A1 and ADAMTS12 in 43 human kidneys, (D) Quantification of ISH. Visualisation of the proportion of ADAMTS12-positive cells that are also PDGFRB-positive. (E) Correlation of ADAMTS12 and PDGFRB-ISH expression in human kidney tissue. (F) Correlation of ADAMTS12 and COL1A1-ISH expression in human kidney tissue.

FIG. 5: Domain structure and organisation of the ADAMTS12 protein. The N-terminus of ADAMTS12 consists of a signalling peptide, a prodomain and a metalloproteinase domain. The C-terminus of ADAMTS12 comprises a disintegrin-like domain, the first thrombospondin type 1 repeat (TSP1), a Cys-rich domain, and 7 additional TSP1 repeats separated by two spacer domains. The second spacer domain is a mucin-like domain (from Wei et al. 2014).

### DETAILED DESCRIPTION OF THE INVENTION

Before describing the invention in detail, it is noted that the present invention is not limited to particular components of the described devices or described steps of the methods, as these methods or devices may vary. It is also noted that the terminology used herein is used only for the purpose of particular embodiments described, and is not intentionally limiting.

It should be noted that in the description and in the appended claims, the simple form such as “a” or “the” implies a singular and/or plural subject matter unless the context clearly dictates otherwise. In the event that a range of parameters has been specified, the limiting numerical values count as limits to the disclosed or claimed numerical range.

It should also be noted that the embodiments disclosed herein are not to be understood as individual embodiments that would not relate to each other. Features discussed in conjunction with one embodiment should also be considered disclosed in conjunction with other embodiments shown herein. If in one case a particular feature is not disclosed with one embodiment but with another, the person skilled in the art will understand that this does not necessarily mean that this feature should not be disclosed with the other embodiment. The person skilled in the art will understand that it is in accordance with the principle of this application to disclose the feature in question also for the other embodiment, but that this has not been done for reasons of clarity and to keep the specification within a manageable scope.

Furthermore, the content of the prior art documents referred to herein is incorporated by reference. This applies in particular to prior art documents that disclose standard or routine methods. In this case, the main purpose of incorporation by reference is to enable sufficient disclosure and to avoid lengthy repetition.

According to a first aspect, the present invention relates to a method for reducing the expression and/or secretion of extracellular matrix (ECM) proteins by a given cell, and/or for inhibiting the migration of fibroblasts, wherein the method comprises at least one step selected from the group consisting of
(i) inhibition or reduction of ADAMTS12 gene expression in the cell,
(ii) inhibition or reduction of ADAMTS12 activity,
(iii) inhibition or reduction of ADAMTS12 protease activity, and/or
(iv) promotion of the degradation of the ADAMTS12 protein.

The inhibition or reduction of ADAMTS12 gene expression may include, for example, ADAMTS12 gene knock-down, knock-out, conditional gene knock-out, gene alteration or mutation, RNA interference, siRNA and/or antisense RNA.

Inhibition or reduction of ADAMTS12 protein activity may involve the use of an active agent that binds to and/or inhibits or reduces the activity of the ADAMTS12 protein (A Disintegrin And Metalloproteinase with ThromboSpondin motifs 12 protein).

Preferably said cell is a renal cell or a cardiac cell, preferably a renal fibroblast cell or a cardiac fibroblast cell, a renal myofibroblast cell or a cardiac myofibroblast cell, or a renal pericyte or cardiac pericyte; most preferably a renal fibroblast cell or a cardiac fibroblast cell.

The ADAMTS12 protein may be a mammalian, non-primate, primate and in particular a human ADAMTS12 protein or a fragment thereof.

According to a second aspect, the present invention relates to a method for identifying an active agent that binds to the ADAMTS12 protein (A Disintegrin And Metalloproteinase with ThromboSpondin motifs 12 protein) or a fragment thereof, and/or inhibits or reduces the activity of the ADAMTS12 protein, or a fragment thereof.

The method comprises at least the following steps:
(i) providing the ADAMTS12 protein or a fragment thereof,
(ii) adding at least one active agent to be analysed for binding to the ADAMTS12 protein or a fragment thereof, and
(iii) identifying the at least one active agent that has bound to the ADAMTS12 protein or a fragment thereof.

Preferably, the active agent to be screened and identified according to the present invention is an ADAMTS12 inhibitor or antagonist, an agent that inhibits or reduces the activity of the ADAMTS12 protein, or a fragment thereof.

The active agent according to the present invention may be selected from the group consisting of a low molecular compound, a natural or synthetic peptide or peptide derivative, and a biologic or biologically active agent.

In the context of the present invention, the term “low molecular compound”, “small molecule” (“smol”) or “chemical drug” refers to an organic compound of low molecular weight (<10,000 daltons, especially <1,000 daltons), often with a size in the order of 1 nm.

Many medicaments are small molecules. Such small molecules can regulate a biological process. Small molecules may be able to inhibit a specific function of a protein. In the field of pharmacology, the term “small molecule” refers specifically to molecules that bind to specific biological macromolecules and act as an effector by altering the activity or function of a target. For example, acetylsalicylic acid (ASA) is considered a low molecular compound, measuring 180 daltons and consisting of 21 atoms. Such low molecular compounds often have little ability to trigger an immune response and remain relatively stable over time.

The low molecular compound according to the present invention may contain, in addition to other chemical backbones, substituents, groups or molecular groups, for example, alkyl, alkenyl, alkynyl, alkoxy, aryl, alkylene, arylene, amino, halogen, carboxylate derivative, cycloalkyl, carbonyl derivative, heterocycloalkyl, heteroaryl, heteroarylene, sulfonate, sulfate, phosphonate, phosphate, phosphine, or phosphine oxide groups.

The “biologic”, “biological drug”, “biological therapeutic agent”, “biopharmaceutical” or “biologically active agent” according to the present invention is preferably an antibody, or an antigen-binding fragment thereof, or an antigen-binding derivative thereof, or an antibody-like molecule or protein, or an aptamer, or a nucleic acid.

In a preferred embodiment of the method for identifying an active agent that binds to the ADAMTS12 protein or a fragment thereof, and/or inhibits or reduces the activity of the ADAMTS12 protein, or a fragment thereof, the active agent is a member of a “library” of compounds.

The “library” (mixture) of compounds may include, for example, low molecular compounds, natural or synthetic peptides or peptide derivatives, or biologics or biological active agents or biological compounds.

In the context of the present invention, the term “(combinatorial) compound library” or “library of compounds” refers to collections of respective chemical compounds, small molecules, natural or synthetic peptides or peptide derivatives, or macromolecules such as proteins or other biologics, each containing a large number of related chemical, peptide or biological species of molecules, respectively, that can be used together in certain screening assays or identification steps.

Methods for producing molecular libraries of low-molecular chemical compounds (“compound libraries”) and for high-throughput screening of the compounds for interaction with the target molecule are described in the prior art (e.g. Volochnyuk et al. 2019). These methods also include so-called “focus libraries”, highly annotated and pre-selected chemical molecule libraries (Wassermann et al. 2014), DNA-encoded libraries of chemical compounds (Martin et al. 2020), and chemoinformatics-based virtual molecule libraries (Saldivar-Gonzalez et al. 2020). The use of phage display technologies to identify suitable small molecule active agents has been described by Takakusagi et al., 2020, for example. Numerous other peptide and antibody display technologies such as bacterial display, yeast surface display, mammalian surface display and ribosome display are described in Valldorf et al., 2022.

Methods for producing molecule libraries, their immobilisation and their high-throughput screening of biological molecules, for example peptides, peptide derivatives, proteins, antibodies, antigen-binding antibody fragments, antigen-binding antibody derivatives or antibody-like molecules, are also described in the prior art (for peptide libraries, for example in Bozovicar and Bratkovic 2019; Schwaar et al. 2019; for antibody libraries in Lin and Lerner 2021).

In a preferred embodiment of the method for identifying an active agent that binds to the ADAMTS12 protein or a fragment thereof, and/or inhibits or reduces the activity of the ADAMTS12 protein or a fragment thereof, the biologic is an antibody, an antigen-binding fragment thereof, an antigen-binding derivative thereof, an antibody-like molecule or protein, an aptamer, or a nucleic acid.

In a preferred embodiment of the method for identifying an active agent that binds to the ADAMTS12 protein or a fragment thereof, and/or inhibits or reduces the activity of the ADAMTS12 protein or a fragment thereof, the ADAMTS12 protein is bound to a solid phase or is present in solution.

According to a third aspect, the present invention relates to the use of a nucleic acid encoding the ADAMTS12 protein or a fragment thereof, or the use of the ADAMTS12 protein or a fragment thereof, in a method for identifying an active agent that binds to the ADAMTS12 protein or a fragment thereof, and/or inhibits or reduces the activity of the ADAMTS12 protein or a fragment thereof.

For expression of the ADAMTS12 metalloproteinase or a fragment thereof, a nucleic acid encoding the ADAMTS12 metalloproteinase or a fragment thereof is cloned into a suitable expression vector, e.g. a suitable expression plasmid, as described (Green and Sambrook 2012). The recombinant expression plasmid is introduced by transfection into a cell suitable for the expression of ADAMTS12 or a fragment thereof, the cell is propagated in cell culture with a suitable cell culture medium, and the expressed protein is purified from the cells and/or the cell culture medium.

As used herein, the term “transfection” refers to any method for intentionally introducing a foreign nucleic acid into a eukaryotic cell. Various types of nucleic acids can be used for transfection into eukaryotic cells, in particular deoxyribonucleic acid (DNA), ribonucleic acid (RNA), as well as small non-coding RNAs such as siRNA, shRNA, and miRNA.

With regard to transfection, a distinction is made between stable and transient transfection. In stable transfection, long-term expression of the transgene is achieved by integrating the nucleic acid introduced into the cell into the cellular genome, while transient transfection, in which the expression of the transgene is only temporary, does not require integration of the nucleic acid into the cellular genome.

The selection of the optimal transfection method depends on various factors, in particular the type and origin of the target or production cell and the type of nucleic acid introduced. Physical, chemical and viral vector-based transfection methods can be used for the introduction of foreign (modified homologous and/or heterologous) nucleic acid encoding the desired transgene into eukaryotic cells. Physical transfection methods include e.g. electroporation, sonoporation, magnetofection, microinjection and biolistic methods. Chemical transfection methods include the calcium phosphate method, the use of dendrimers, cationic polymers such as diethylaminoethyl-dextran (DEAE-dextran), nanoparticles, non-liposomal nanoparticles, and liposomal transfection. Transfection using viral vectors (so-called “transduction”) involves the use of genetically modified retro- and lentiviruses, adenoviruses and adeno-associated viruses (AAV) in particular (Fus-Kujawa et al. 2021).

According to a fourth aspect, the present invention relates to an active agent obtained by said method for identifying an active agent that binds to the ADAMTS12 protein, or a fragment thereof, and/or inhibits or reduces the activity of the ADAMTS12 protein, or a fragment thereof, or obtained by any of the embodiments of said method described above.

Furthermore, the present invention relates to an active agent that binds to the ADAMTS12 protein or a fragment thereof, and/or inhibits or reduces the activity of the ADAMTS12 protein, or a fragment thereof, and/or promotes the degradation of the ADAMTS12 protein.

Furthermore, the present invention relates to an active agent which inhibits or reduces the expression of the ADAMTS12 gene in a renal cell or a cardiac cell, preferably wherein the renal cell is a renal fibroblast cell and/or the cardiac cell is a cardiac fibroblast cell.

In a preferred embodiment, the present invention relates to an active agent, wherein the active agent is a low molecular compound (smol), a peptide or peptide derivative, or a biologic, preferably wherein the biologic is an antibody or an antigen-binding fragment thereof, or an antigen-binding derivative thereof, or an antibody-like protein, or an aptamer or a nucleic acid.

In a preferred embodiment, the active agent binds specifically with a high or particularly high affinity and/or avidity to the ADAMTS12 protein or a fragment thereof. In a preferred embodiment, the active agent, when bound to ADAMTS12, reduces or inhibits ADAMTS12 activity.

The term “specifically bind” as used herein means that the active agent has a dissociation constant KD with respect to its binding to the ADAMTS12 protein molecule or an epitope thereof of about 100 μM or less. In one embodiment, the KD is about 100 μM or lower, about 50 μM or lower, about 30 μM or lower, about 20 μM or lower, about 10 μM or lower, about 5 μM or lower, about 1 μM or lower, about 900 nM or lower, about 800 nM or lower, about 700 nM or lower, about 600 nM or lower, about 500 nM or lower, about 400 nM or lower, about 300 nM or lower, about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, or about 10 nM or lower, about 1 nM or lower, about 900 μM or lower, about 800 μM or lower, about 700 μM or lower, about 600 μM or lower, about 500 μM or lower, about 400 μM or lower, about 300 μM or lower, about 200 μM or lower, about 100 μM or lower, about 90 μM or lower, about 80 μM or lower, about 70 μM or lower, about 60 μM or lower, about 50 μM or lower, about 40 μM or lower, about 30 μM or lower, about 20 μM or lower, or about 10 μM or lower, or about 1 μM or lower.

According to a fifth aspect, the present invention relates to an antibody, or an antigen-binding fragment or antigen-binding derivative thereof, or an antibody-like protein that specifically binds to the ADAMTS12 protein.

In a preferred embodiment, the present invention relates to said antibody, or antigen-binding fragment or antigen-binding derivative thereof, or antibody-like protein, wherein the antibody, or antigen-binding fragment or derivative thereof, or antibody-like protein inhibits ADAMTS12 activity, i.e. acts as an inhibitor or antagonist of ADAMTS12.

As used herein, the term “antibody” refers to a protein consisting of one or more polypeptide chains encoded by immunoglobulin genes or fragments of immunoglobulin genes or cDNAs derived therefrom. These immunoglobulin genes include the light chain genes kappa, lambda and the heavy chain genes alpha, delta, epsilon, gamma and mu of the constant region as well as each of the many different genes of the variable region.

The basic structural unit of immunoglobulin (antibody) is usually a tetramer consisting of two identical pairs of polypeptide chains, the light chains (L, with a molecular weight of about 25 kDa) and the heavy chains (H, with a molecular weight of about 50-70 kDa). Each heavy chain consists of a variable region of the heavy chain (abbreviated as VH or V_{H}) and a constant region of the heavy chain (abbreviated as CH or C_{H}). The heavy chain constant region consists of three domains, namely CH1, CH2 and CH3. Each light chain contains a variable light chain region (abbreviated as VL or V_{L}) and a constant light chain region (abbreviated as CL or C_{L}). The VH and VL regions can be further subdivided into regions of hypervariability, also known as complementarity determining regions (CDR), interspersed with regions that are more conserved, known as framework regions (FR). Each VH and VL region consists of three CDRs and four FRs arranged from the amino terminus to the carboxy terminus in the order FR1, CDR1, FR2, CDR2, FR3, CDR3, FR4. The variable regions of the heavy and light chains form a binding domain that interacts with an antigen.

The CDRs are most important for the binding of the antibody or the antigen-binding part thereof. The FRs can be replaced by other sequences as long as the three-dimensional structure required for binding the antigen is retained.

The term “antigen-binding part” of the (monoclonal) antibody refers to one or more fragments of an antibody that retain the ability to specifically bind to the antigen in its native form. Examples of antigen-binding parts of the antibody include a Fab fragment, a monovalent fragment consisting of the VL, VH, CL and CH1 domains, an F(ab′)2 fragment, a bivalent fragment comprising two Fab fragments joined by a disulfide bridge at the hinge region, an Fd fragment consisting of the VH and CH1 domains, an Fv fragment consisting of the VL and VH domains of a single arm of an antibody, and a dAb fragment consisting of a VH domain and an isolated complementarity-determining region (CDR).

The antibody, antibody fragment or antibody derivative thereof according to the present invention may be a monoclonal antibody. The antibody may be of the IgA, IgD, IgE, IgG or IgM isotype.

As used herein, the term “monoclonal antibody (mAb)” refers to an antibody composition having a homogeneous antibody population, i.e. a homogeneous population consisting of a whole immunoglobulin or a fragment or derivative thereof. Particularly preferred is such an antibody selected from the group consisting of IgG, IgD, IgE, IgA and/or IgM, or a fragment or derivative thereof.

As used herein, the term “fragment” refers to fragments of such an antibody that retain their target binding capacities, e.g., a CDR (complementarity-determining region), a hypervariable region, a variable domain (Fv), a heavy IgG chain (consisting of VH, CH1, hinge, CH2 and CH3 regions), a light IgG chain (consisting of VL and CL regions) and/or a Fab and/or F(ab)2.

As used herein, the term “derivative” refers to protein constructs that are structurally different from the common antibody concept but still have some structural relationship to it, e.g. scFv, Fab and/or F(ab)2, as well as bi-, tri- or higher-specific antibody constructs. All these elements are explained below.

Other antibody derivatives known to the person skilled in the art are diabodies, camelid antibodies, domain antibodies, bivalent homodimers with two chains consisting of scFvs, IgAs (two IgG structures linked by a J-chain and a secretory component), Hai antibodies, antibodies consisting of new world primates scaffold plus non-new world primates CDR, dimerised constructs comprising CH3+VL+VH, other scaffold protein formats comprising CDRs, and antibody conjugates.

As used herein, the term “antibody-like protein” refers to a protein that has been modified (e.g. by mutagenesis of Ig loops) to bind specifically to a target molecule. Typically, such an antibody-like protein comprises at least one variable peptide loop that is bound to a protein scaffold at both ends. This double structural constraint increases the binding affinity of the antibody-like protein to a level comparable to that of an antibody. The length of the variable peptide loop typically consists of 10 to 20 amino acids. The scaffold protein can be any protein with good solubility properties. Preferably, the scaffold protein is a small globular protein. Antibody-like proteins include, without limitation, affibodies, anticalins and designed ankyrin proteins and affilin proteins. Antibody-like proteins can be derived from large libraries of mutants, e.g. by panning from large phage display libraries, and can be isolated by analogy with regular antibodies. Antibody-like binding proteins can also be obtained by combinatorial mutagenesis of surface-exposed groups in globular proteins. Antibody-like proteins have been described, for example, in Binz et al. (2005) and Hosse et al. (2006).

As used herein, the term “Fab” refers to an IgG fragment comprising the antigen binding region, wherein the fragment is composed of a constant and a variable domain of the heavy and light chain of the antibody, respectively.

As used herein, the term “F(ab)2” refers to an IgG fragment consisting of two Fab fragments linked by disulfide bonds.

The term “scFv” as used herein refers to a variable single chain fragment that is a fusion of the variable regions of the heavy and light chains of immunoglobulins joined by a short linker, usually comprising serine (S) and/or glycine (G) groups. This chimeric molecule retains the specificity of the original immunoglobulin, despite the removal of the constant regions and the introduction of a linker peptide.

Modified antibody formats are, for example, bi- or trispecific antibody constructs, antibody-based fusion proteins, immunoconjugates and the like.

IgG, scFv, Fab and/or F(ab)2 are antibody formats that are well known to the person skilled in the art. Detailed explanations and techniques can be found in the relevant textbooks.

According to preferred embodiments of the present invention, the antibody or the antigen-binding fragment thereof or the antigen-binding derivative thereof is a murine, a chimeric, a humanised or a human antibody or an antigen-binding fragment or an antigen-binding derivative thereof.

Mouse-derived monoclonal antibodies (mAb) can cause undesirable immunological side effects because they contain a protein from another species that can induce an immune response. To overcome this problem, antibody humanisation and maturation methods have been developed to generate antibody molecules with minimal immunogenicity when used in humans, while ideally preserving the specificity and affinity of the non-human parent antibody. In these methods, for example, the scaffold regions of a mouse mAb are replaced by corresponding human scaffold regions (so-called CDR grafting). WO200907861 discloses the generation of humanised forms of mouse antibodies by linking the CDR regions of non-human antibodies with human constant regions using recombinant DNA technology.

U.S. Pat. No. 6,548,640 describes CDR transplantation techniques, and U.S. Pat. No. 5,859,205 describes the production of humanised antibodies.

As used herein, the term “humanised antibody” refers to an antibody, fragment or derivative thereof in which at least a portion of the constant regions and/or scaffold regions and optionally a portion of the CDR regions of the antibody are derived from or adapted to human immunoglobulin sequences.

According to a sixth aspect, the present invention relates to an active agent as described above or an antibody, an antigen-binding fragment or an antigen-binding derivative thereof, or an antibody-like protein as described above, for use in the treatment of a chronic kidney disease and/or a heart disease.

The chronic kidney disease is preferably progressive chronic renal insufficiency and/or renal fibrosis in this case. The heart disease is preferably heart failure, myocardial infarction and/or cardiac fibrosis.

Furthermore, the present invention relates to a pharmaceutical composition comprising the active agent as described above, or the antibody, antigen-binding fragment or antigen-binding derivative thereof, or an antibody-like protein as described above, and one or more pharmaceutically acceptable excipients, for use in the treatment of a chronic kidney disease and/or a heart disease, preferably wherein the chronic kidney disease is a progressive chronic kidney disease, a renal insufficiency and/or renal fibrosis, and preferably wherein the heart disease is a heart failure and/or cardiac fibrosis.

In a preferred embodiment of the present invention, said pharmaceutically acceptable excipient(s) is/are selected from the group consisting of pharmaceutically acceptable buffers, surfactants, diluents, carriers, excipients, fillers, binders, lubricants, slip additives, disinfectants, adsorbents and/or preservatives.

Said pharmaceutical composition may be administered in the form of powder, tablets, pills, capsules or beads. In aqueous form, the pharmaceutical formulation may be ready for administration, whereas the formulation in lyophilised form needs to be converted into a liquid form prior to administration, for example by adding water for injection containing, or not, a preservative such as, but not limited to, benzyl alcohol, antioxidants such as vitamin A, vitamin E, vitamin C, vitamin D, benzyl alcohol, antioxidants such as vitamin A, vitamin E, vitamin C, retinyl palmitate and selenium, the amino acids cysteine and methionine, citric acid and sodium citrate, synthetic preservatives such as the parabens methylparaben and propylparaben.

The pharmaceutical formulation may further comprise one or more stabilisers, which may be, for example, an amino acid, a sugar polyol, a disaccharide and/or a polysaccharide. The pharmaceutical formulation may further comprise one or more surfactants, one or more isotonising agents and/or one or more metal ion chelators and/or one or more preservatives.

The pharmaceutical formulation as described herein may be suitable for at least oral, parenteral, intravenous, intramuscular or subcutaneous administration. Alternatively, the active agent or antibody according to the present invention may be provided in a sustained release formulation that allows for the sustained release of the active agent over a period of time.

There is further provided a primary packaging, such as a prefilled syringe or pen, vial or infusion bag, comprising said pharmaceutical formulation according to this aspect of the invention.

The prefilled syringe or pen may contain the formulation either in lyophilised form (which must then be dissolved with water for injection, for example, before administration) or in aqueous form. The syringe or pen is often a single-use disposable and can have a volume of between 0.1 and 20 ml. However, the syringe or pen can also be a reusable syringe or a multi-dose pen.

Furthermore, the present invention relates to the use of an active agent that binds to the ADAMTS12 protein in a method for treating a chronic kidney disease and/or a heart disease, wherein the chronic kidney disease is preferably a progressive chronic kidney disease, a renal insufficiency and/or renal fibrosis, and/or wherein the heart disease is preferably a heart failure and/or cardiac fibrosis. Preferably, when bound to ADAMTS12, the active agent inhibits ADAMTS12 activity.

Furthermore, the present invention relates to the use of an active agent which binds to the ADAMTS12 protein for the production of a drug for the treatment of a chronic kidney disease and/or a heart disease, wherein the chronic kidney disease is preferably progressive chronic renal insufficiency and/or renal fibrosis, and wherein the heart disease is preferably heart failure and/or cardiac fibrosis. Preferably, when bound to ADAMTS12, the active agent inhibits ADAMTS12 activity.

Furthermore, the present invention relates to a method for treating or preventing a chronic kidney disease and/or a heart disease, wherein the method comprises administering to a human or animal subject an active agent that binds to and/or inhibits the ADAMTS12 protein in a therapeutically effective dose or amount.

As used herein, the term “effective dose” or “effective amount” means a dose or amount of the active agent that is necessary, in terms of dosages and administration times, to achieve the desired therapeutic result in a patient. Effective amounts may vary depending on factors such as the disease state, the age, gender and/or weight of the patient, the pharmaceutical formulation, the subtype of disease being treated, and the like, but may nevertheless be routinely determined by the person skilled in the art.

According to a seventh aspect, the present invention relates to a method for producing an active agent according to the method for identifying said active agent which binds to the ADAMTS12 protein or a fragment thereof, and/or inhibits or reduces the activity of the ADAMTS12 protein, or a fragment thereof, as described above, further comprising purifying said active agent.

The present invention further relates to a method for producing a pharmaceutical composition comprising
(i) the method of identifying said active agent that binds to the ADAMTS12 protein or a fragment thereof, and/or inhibits or reduces the activity of the ADAMTS12 protein, or a fragment thereof, as described above, and further
(ii) mixing the identified active agent with a pharmaceutically acceptable carrier.

According to an eighth aspect, the present invention relates to a composition comprising a combination of
(i) the active agent which binds to the ADAMTS12 protein or a fragment thereof, and/or inhibits or reduces the activity of the ADAMTS12 protein, or a fragment thereof, as described above, or the antibody or antigen-binding fragment or antigen-binding derivative thereof, or antibody-like protein as described above, or the pharmaceutical composition comprising the active agent as described above, or the antibody, antigen-binding fragment or antigen-binding derivative thereof, or antibody-like protein as described above, and one or more pharmaceutically acceptable excipients, and
(ii) one or more other therapeutically active compounds.

According to a ninth aspect, the present invention relates to a therapeutic kit comprising:
(i) the pharmaceutical composition as described above,
(ii) a device for administering said composition, and
(iii) optionally, instructions for use.

### Sequences

TABLE 1
Human ADAMTS12, amino acid sequence (UniProt-ID: P58397-1)

| SEQ ID No. | Sequence |
|---|---|
| SEQ ID No. 1 | 10 20 30 40 50 |
| | MPCAQRSWLA NLSVVAQLLN FGALCYGRQP QPGPVREPDR RQEHFIKGLP |
| | 60 70 80 90 100 |
| | EYHVVGPVRV DASGHFLSYG LHYPITSSRR KRDLDGSEDW VYYRISHEEK |
| | 110 120 130 140 150 |
| | DLFFNLTVNQ GFLSNSYIME KRYGNLSHVK MMASSAPLCH LSGTVLQQGT |
| | 160 170 180 190 200 |
| | RVGTAALSAC HGLTGFFQLP HGDFFIEPVK KHPLVEGGYH PHIVYRRQKV |
| | 210 220 230 240 250 |
| | PETKEPTCGL KDSVNISQKQ ELWREKWERH NLPSRSLSRR SISKERWVET |
| | 260 270 280 290 300 |
| | LVVADTKMIE YHGSENVESY ILTIMNMVTG LFHNPSIGNA IHIVVVRLIL |
| | 310 320 330 340 350 |
| | LEEEEQGLKI VHHAEKTLSS FCKWQKSINP KSDLNPVHHD VAVLLTRKDI |
| | 360 370 380 390 400 |
| | CAGFNRPCET LGLSHLSGMC QPHRSCNINE DSGLPLAFTI AHELGHSFGI |
| | 410 420 430 440 450 |
| | QHDGKENDCE PVGRHPYIMS RQLQYDPTPL TWSKCSEEYI TRFLDRGWGF |
| | 460 470 480 490 500 |
| | CLDDIPKKKG LKSKVIAPGV IYDVHHQCQL QYGPNATFCQ EVENVCQTLW |
| | 510 520 530 540 550 |
| | CSVKGFCRSK LDAAADGTQC GEKKWCMAGK CITVGKKPES IPGGWGRWSP |
| | 560 570 580 590 600 |
| | WSHCSRTCGA GVQSAERLCN NPEPKFGGKY CTGERKRYRL CNVHPCRSEA |
| | 610 620 630 640 650 |
| | PTFRQMQCSE FDTVPYKNEL YHWFPIFNPA HPCELYCRPI DGQFSEKMLD |
| | 660 670 680 690 700 |
| | AVIDGTPCFE GGNSRNVCIN GICKMVGCDY EIDSNATEDR CGVCLGDGSS |
| | 710 720 730 740 750 |
| | CQTVRKMFKQ KEGSGYVDIG LIPKGARDIR VMEIEGAGNF LAIRSEDPEK |
| | 760 770 780 790 800 |
| | YYLNGGFIIQ WNGNYKLAGT VFQYDRKGDL EKLMATGPTN ESVWIQLLFQ |
| | 810 820 830 840 850 |
| | VTNPGIKYEY TIQKDGLDND VEQQMYFWQY GHWTECSVTC GTGIRRQTAH |
| | 860 870 880 890 900 |
| | CIKKGRGMVK ATFCDPETQP NGRQKKCHEK ACPPRWWAGE WEACSATCGP |
| | 910 920 930 940 950 |
| | HGEKKRTVLC IQTMVSDEQA LPPTDCQHLL KPKTLLSCNR DILCPSDWTV |
| | 960 970 980 990 1000 |
| | GNWSECSVSC GGGVRIRSVT CAKNHDEPCD VTRKPNSRAL CGLQQCPSSR |
| | 1010 1020 1030 1040 1050 |
| | RVLKPNKGTI SNGKNPPTLK PVPPPTSRPR MLTTPTGPES MSTSTPAISS |
| | 1060 1070 1080 1090 1100 |
| | PSPTTASKEG DLGGKQWQDS STQPELSSRY LISTGSTSQP ILTSQSLSIQ |
| | 1110 1120 1130 1140 1150 |
| | PSEENVSSSD TGPTSEGGLV ATTTSGSGLS SSRNPITWPV TPFYNTLTKG |
| | 1160 1170 1180 1190 1200 |
| | PEMEIHSGSG EEREQPEDKD ESNPVIWTKI RVPGNDAPVE STEMPLAPPL |
| | 1210 1220 1230 1240 1250 |
| | TPDLSRESWW PPFSTVMEGL LPSQRPTTSE TGTPRVEGMV TEKPANTLLP |
| | 1260 1270 1280 1290 1300 |
| | LGGDHQPEPS GKTANRNHLK LPNNMNQTKS SEPVLTEEDA TSLITEGFLL |
| | 1310 1320 1330 1340 1350 |
| | NASNYKQLTN GHGSAHWIVG NWSECSTTCG LGAYWRRVEC STQMDSDCAA |
| | 1360 1370 1380 1390 1400 |
| | IQRPDPAKRC HLRPCAGWKV GNWSKCSRNC SGGFKIREIQ CVDSRDHRNL |
| | 1410 1420 1430 1440 1450 |
| | RPFHCQFLAG IPPPLSMSCN PEPCEAWQVE PWSQCSRSCG GGVQERGVFC |
| | 1460 1470 1480 1490 1500 |
| | PGGLCDWTKR PTSTMSCNEH LCCHWATGNW DLCSTSCGGG FQKRTVQCVP |
| | 1510 1520 1530 1540 1550 |
| | SEGNKTEDQD QCLCDHKPRP PEFKKCNQQA CKKSADLLCT KDKLSASFCQ |
| | 1560 1570 1580 1590 |
| | TLKAMKKCSV PTVRAECCFS CPQTHITHTQ RQRRQRLLQK SKEL |

### EXAMPLES

The present invention is explained in more detail by the examples and drawings shown and discussed below. It should be noted that the examples and drawings are descriptive only and are not intended to limit the invention in any way.

The invention is not limited to the disclosed embodiments. Other variations of the disclosed embodiments may be understood and carried out by those skilled in the art from a study of the drawings, the disclosure, and the appended claims in carrying out the claimed invention. In the claims, the word “comprising” does not exclude other elements or steps, and the indefinite article “a” or “one” does not exclude a plurality. The mere fact that certain measures are recited in different dependent claims does not mean that a combination of these measures cannot be advantageously used. Any reference signs in the claims are not to be understood as limiting the scope of application.

All amino acid sequences disclosed here are shown from N-terminus to C-terminus; all nucleic acid sequences disclosed here are shown 5′->3′.

### Example 1: Material and Methods

### Mice:

GlilCreER^{t2} (JAX Stock #007913) and Rosa26tdTomato (JAX Stock #007909) were purchased from Jackson Laboratories (Bar Harbor, ME, USA). The progeny were genotyped by PCR according to the Jackson Laboratories protocol. ADAMTS12-KO mice were developed by C. Lopez-Otin (El Hour et al., 2010). Genotyping of all mice was performed by PCR. The mice were kept under specific pathogen-free conditions at RWTH Aachen University.

### Treatment of the Mice:

For unilateral ureteral obstruction (UUO), the left ureter was ligated at the level of the lower pole with two 7.0 bands (Ethicon) after a flank incision. For ischaemia-reperfusion surgery (IRI), the renal artery was clamped with an aneurysm clamp for 26 minutes after a flank incision. An isolated flank incision was made for placebo surgery (Sham). The mice were sacrificed on day 10 after unilateral ureteral surgery or day 28 after ischaemia-reperfusion surgery. The animal experiment protocols were approved by the State Office for Nature, Environment and Consumer Protection of North Rhine-Westphalia (Germany). All animal experiments were performed in accordance with their guidelines. For inducible fate-tracking, Gli1CreER;tdTomato mice (8 weeks old) received tamoxifen three times by gavage (10 mg p.o.). The administration of tamoxifen leads to the translocation of Cre recombinase into the cell nucleus in Gli1-expressing cells, which cuts the loxP DNA sequences. Recombination removes a stop codon and the underlying fluorophore tdTomato is expressed in Gli1-expressing cells. This results in genetic labelling of Gli1-positive cells after administration of tamoxifen. 21 days after tamoxifen administration, a UUO or sham surgery was performed and 10 days after the operation the mice were killed. Killed mice were perfused via the left heart with 20 ml of 0.9% NaCl to remove residual blood from the vascular system.

Myocardial infarction and myocardial infarction sham surgery were performed as previously described (Curaj et al., 2015). In summary, mice were anaesthetised with isoflurane (2-2.5%), intubated and ventilated with oxygen using a mouse ventilator (Harvard Apparatus, March, Germany). For analgesia, metamizole was injected subcutaneously (200 μg/g bw), in addition to local analgesia by subcutaneous and intercostal injection of bupivacaine (2.5 μg/g bw). After a left-sided thoracotomy, either a sham myocardial infarction surgery (no intervention) or a myocardial infarction surgery by ligation of the anterior interventricular ramus (RIVA) with a silk suture (0-7) was performed. The ribs, the muscle layer and the skin incision were then sutured with Prolene (0-6). Postoperative analgesia was administered for three days using metamizole in drinking water (1.25 mg/ml in 1% sucrose).

### Single Cell Isolation and Fluorescence-Activated Cell Sorting (FACS):

The kidneys were surgically removed, cut into small slices and placed in a 15-ml tube (Falcon) on ice-cold phosphate-buffered saline with 1% foetal calf serum (PBS with 1% FBS). The kidney tissue was then transferred to a C-tube (Miltenyi Biotec) and processed on a gentle-MACS (Miltenyi Biotec) using the Spleen 4 programme. The tissue was digested for 30 min at 37° C. with shaking at 300 RPM in a digestion solution containing 25 μg/ml Liberase TL (Roche) and 50 μg/ml DNase (Sigma) in RPMI (Gibco). After incubation, the samples were processed again on a gentle-MACS (Miltenyi Biotec) using the same programme. The resulting suspension was passed through a 70 μm cell strainer (Falcon), washed with 45 ml cold PBS and centrifuged at 500 g for 5 minutes at 4° C. The cells were counted using a haemocytometer with trypan blue staining. Overall viability was over 80% using this method. The isolated cells were resuspended in PBS with 1% FBS on ice at a final concentration of 1×10⁷ cells/ml. Live, single cells were isolated by FACS sorting using a FACS Aria II device (Becton Dickinson, Basel, Switzerland) and gating on Gli1-tdTomato positive, DAPI-negative cells. On average, it took 5-6 hours from collection of the biopsies to preparation of the single cell suspensions.

### Analysis of Affymetrix Microarray Data:

Microarray gene expression was quantified using the R package “affy” for the mouse genome “Mouse4302.db” and normalised using Robust-Multichip Average (RMA-). The R package Limma (v.3.44.1) was used to test differential gene expression between UUO and placebo surgery (Sham) using the RunLimma function. When microarray samples were mapped to the same gene multiple times, duplicate genes were removed. Differentially expressed genes were ranked according to their T-value. For pathway analysis, the R package “fgsea” was used with the Hallmark signalling pathways based on all differentially expressed genes.

### RNA In Situ Hybridisation:

In situ hybridisation (ISH) was performed using formalin-fixed, paraffin-embedded tissue samples and the RNAScope Multiplex Detection KIT V2 (RNAScope, #323100) according to the manufacturer's protocol with minor modifications. Antigen retrieval was performed for 30 min. 3-5 drops of pretreatment 1 solution were incubated at RT for 10 min after antigen retrieval was performed. The washing steps were performed three times for 5 minutes. The following probes were used for the RNAscope assay: Mm-Pdgfrb #411381-C3, Mm-Adamts12 #400531, Hs-PDGFRß #548991-C1, Hs-COL1A1 #401891-C2, HsADAMTS12 #509701-C3.

### Confocal Imaging:

Images were captured with a Nikon A1R confocal microscope using 40× and 60× objectives (Nikon). The raw data were processed with Nikon software or ImageJ.

### Image Quantification—ISH Image Analysis:

A systematic random selection of the renal cortex was made to select at least 7 representative tubulointerstitial areas per image. Images were split into RGB channels using ImageJ, the background subtracted (rolling ball radius: 10.0 pixels) and fluorescent dots (transcripts) counted. For cell classification, 3 representative Z-stacks (a Z-stack refers to the acquisition of several images using a confocal microscope, which are taken at a certain distance between the first and last focal plane of the same region) were taken from each sample. The Z-stacks were superimposed as so-called Z-projects and divided into RGB channels using ImageJ. The cells were segmented and classified with a trained algorithm using the ilastik object classification workflow (Berg et al., 2019).

### Quantitative RT-PCR:

For RNA extraction from cultured cells, the cells were washed with PBS and then lysed with RNA-Easy Lysis Buffer. For RNA extraction from kidney tissue, the tissue was transferred to an Eppendorf tube containing 400 μl RNA-Easy Lysis Buffer and digested using Mixer-Mill (2×2 min, 20 Hz). RNA extraction was then performed according to the manufacturer's instructions using the RNeasy Mini Kit (giagen). 200 ng of RNA were reverse transcribed using the High-Capacity cDNA Reverse Transcription Kit (Applied Biosystems). The qRT-PCR was performed using the iTaq Universal SYBR Green Supermix (Biorad) and the Bio-Rad CFX96 Real Time System with the C1000 Touch thermal cycler. The cycling conditions were: 95° C. for 3 minutes, then 40 cycles of 95° C. for 15 seconds and 60° C. for 1 minute, followed by one cycle of 95° C. for 10 seconds. GAPDH was used as the housekeeping gene. The data were analysed using the 2-CT method. The primers used are listed in Table 2.

TABLE 2
List of RT-PCR primer sequences (human)

| Gene | Forward and Reverse Primer |
|---|---|
| murine Gapdh | Fw 5′-AAGTGGTGATGGGCTTCCC-3′ |
| | Rv 5′-GGCAAATTCAACGGCACAGT-3′ |
| murine Adamts12 | Fw 5′-GTGTGGAACAGGTATCCG-3′ |
| | Rv 5′-CAGAGCTCGACTGTTGGG-3′ |
| murine Col1a1 | Fw 5′-TGACTGGAAGAGCGGAGAGT-3′ |
| | Rv 5′-GTTCGGGCTGATGTA-3′ |
| murine Fn | Fw 5′-ATCTGGACCCCTCCT-3′ |
| | Rv 5′-GCCCAGTGATTTCAG-3′ |
| Human GAPDH | Fw 5′-GAAGGTGAAGGTCGGAGTCA-3′ |
| | Rv 5′-TGGACTCCACGACGTACTCA-3′ |
| Human ADAMTS12 | Fw 5′-GTGTGGAACAGGTATCCG-3′ |
| | Rv 5′-CAGAGCTCGACTGTTGGG-3′ |
| Human COL1A1 | Fw 5′-CCCAGCCACAAAGAGTCTACA-3′ |
| | Rv 5′-ATTGGTGGGATGTCTTCGTCT-3′ |

### Immunofluorescence Staining Including Quantification:

Using 2-μm paraffin-embedded, formalin-fixed kidney sections, slides were blocked in 10% bovine serum, followed by incubation of the primary antibody for 1 hour, washing 3 times for 5 minutes in PBS and subsequent incubation of the secondary antibodies for 30 minutes. After DAPI (4′,6‘-’diamidino-2-phenylindole) staining (Roche, 1:10,000), the slides were mounted with ImmuMount (9990402, Epredia). Four representative images of the renal cortex were taken per sample using the 40× objective of a Nikon AIR confocal microscope. For quantification, the images were divided into RGB channels and fluorescent areas were quantified using ImageJ. The following antibodies were used: anti-mouse PDGFRP (ab32570, 1:100, Abcam), AF488 donkey anti-rabbit (711-545-152, 1:200, Jackson ImmunoResearch), AF647 donkey anti-rat (712-605-153, 1:200, Jackson ImmunoResearch).

### Immunohistochemistry Including Quantification:

After deparaffinisation of 2 μm paraffin sections, antigen retrieval was performed by heating the sections three times for 5 minutes in an antigen unmasking solution (H-33000, Vector Laboratories). This was followed by a 3-minute incubation with 3% hydrogen peroxide and then an incubation with avidin/biotin (VEC-SP-2001, Vector Laboratories) for 10 minutes, followed by a one-hour incubation with the primary antibody, three washes in PBS and subsequent incubation with the secondary antibody. Detection was performed using the DAB substrate kit (SK-4100, Vector Laboratories). Finally, the sections were counterstained with haematoxylin, dehydrated and coverslipped. From each section, 7 representative images of the renal cortex were taken with the 40× objective of a brightfield microscope (BZ-9000, Keyence, IHC). The following antibodies were used: Anti-mouse Coll (1310-01, 1:100, Southern Biotech), biotinylated horse anti-goat antibody (BA-9500, 1:300, Vector Laboratories).

### Mouse Echocardiography

Left ventricular cardiac function was measured using a small animal ultrasound scanner (Vevo 3100 and MX550D transducer, FUJIFILM Visualsonics, Toronto, Ontario, Canada) two days before, four weeks after, and eight weeks after myocardial infarction. Measurements of the short and long cardiac axes, left ventricular end-diastolic and end-systolic volumes and heart rate were performed in B-mode (2D real time) and M-mode with a 40 MHz transducer (MX550D). During the procedure, the mice were anaesthetised with 1-2% isoflurane. All measurements were analysed using VevoLab software.

### Picrosirius Red Colouring and Quantification:

Picro-Sirius red staining was performed using the Morphisto-Sirius red staining kit (13425, Morphisto). Whole slides were scanned with the Aperio Slide Scanner (Leica Biosystems) and fibrotic areas stained red by the Picrosirius kit were quantified using the Aperio eSlide Manager programme.

Processing of Human Tissue: Human kidney tissue was harvested from normal regions as previously described (Kuppe et al., 2021). Tissue was frozen on dry ice or placed in pre-cooled University of Wisconsin solution (#BTLBUW, Bridge to Life Ltd., Columbia, U.S.) and transported on ice to the laboratory. To isolate single kidney cells, a combination of enzymatic and mechanical disruption was used as described above for the isolation of mouse single cells.

FACS of Human Tissue: The isolated cells were stained and isolated as previously described (Kuppe et al., 2021). In summary, the isolated cells were resuspended in 1% PBS-FBS on ice at a final concentration of 1×10⁷ cells/ml. Cells were pre-incubated with Fc block (TruStainFx human, TruStainFx mouse clone 91, biolegend) and then incubated with the anti-CD10 human antibody (clone HI10a, biolegend) diluted in 2% FBS/PBS for 30 minutes on ice protected from light. For staining with human anti-PDGFRb, goat anti-mouse Dyelight 405 (poly24091, biolegend) was used as secondary antibody. All compensations were performed at the time of acquisition using single colour staining and negative staining and fluorescence minus one controls. Individual cells were enriched by FACS sorting and gating to DAPI-negative cells with further enrichment of fibroblasts by PDGFRB staining. Cells were sorted in semi-purity mode aiming for >80% efficiency using the SONY SH800 sorter (Sony Biotechnology; 100 um nozzle sorting chip Sony).

### 10× Genomics 3′ Sc-RNA-Seq (V2 and V3) Single Cell Assays:

Single cell assays were performed as previously described (Kuppe et al., 2021). In summary, a single cell solution of primary human kidney cells was loaded onto a Chromium Single Cell Chip Kit and libraries were processed using the Chromium Single Cell 3′ Library Kit V2 and the i7 Multiplex Kit (PN-120236, PN-120237, PN-120262, 10× Genomics) according to the manufacturer's protocol. The quality of the library was determined using the D1000 ScreenTape on the 2200 TapeStation system (Agilent Technologies). Sequencing was performed on an Illumina Novaseq platform with S1 and S2 flow cells (Ilumina).

Microarray from Human Kidney Tissue:

Paraffin-embedded kidney microarrays were prepared as previously described (Kuppe et al., 2021). In summary, paraffin-embedded, formalin-fixed kidney samples from the Biomaterialbank Aachen were selected based on a previously performed PAS staining. Areas were randomly selected per sample and a 2-mm core was taken from each kidney sample using the TMArrayer™ (Pathology Devices, Beecher Instruments, Westminster, USA). Each core was placed in a receiver block in a 2 mm grid covering approximately 2.5 cm² and sections 5 micron thick were cut and processed using standard histological techniques.

### Generation of a Human PDGFRb+ Cell Line:

An immortalised, renal human PDGFRb-positive cell line was used for in vitro experiments. The generation of the cell line was described in previous work (Kuppe et al., 2021)

### TGFb Treatment Experiments:

TGFb (100-21-10UG, Peprotech) at a concentration of 10 ng/ml in PBS was added to 75% confluent PDGFRb cells after incubation in starvation medium (0.5% foetal serum-containing medium) for 24 hours.

sgRNA:CRISPR-Cas9 Vector Construction, Virus Production and Transduction:

The ADAMTS 12-specific guide RNA
(forwards 5′-CACCGAACATCATAGATCACTCCGG-3′;
backwards 5′-AAACCCGGAGTGATCTATGATGTTC-3)

were cloned into the pL-CRISPR.EFS.GFP plasmid (Addgene #57818) using BsmBI restriction digestion. Lentiviral particles were produced by transient co-transfection of HEK293T cells with the lentiviral transfer plasmid, the packaging plasmid psPAX2 (Addgene #12260) and the VSVG packaging plasmid pMD2.G (Addgene #12259) using TransIT-LT (Mirus). Viral supernatants were collected 48-72 hours after transfection, clarified by centrifugation, supplemented with 10% FCS and Polybrene (Sigma-Aldrich, final concentration of 8 μg/ml) and filtered through a 0.45 μm sieve (Millipore; SLHP033RS). Cell transduction was performed by incubating the PDGFRß cells with viral supernatants for 48 h. eGFP-expressing cells were individually sorted into 96-well plates. To determine mutational events on both alleles within the cultured clones, the PCR product of the ADAMTS12 clones was subcloned into the pCR™ 4Blunt-TOPO vector (Thermo Scientific K287520). At least 6 colonies per CRISPR clone were grown and sequenced (clone C2: 30 colonies were sequenced). At the same time, qPCR was performed to confirm the loss of ADAMTS12 gene expression.

### Retroviral Overexpression of ADAMTS12:

The construction of the ADAMTS12 vector and generation of stable ADAMTS12-overexpressing cell lines was performed as follows. The human cDNA of ADAMTS12 was synthesised by combining two gblock gene fragments (IDT) ([1] Xho-N-terminus-EcoRI and [2] EcoRI-C-terminus-1×HA-tag-EcoRI) and fused to a continuous CDS with a C-terminal 1×HA tag in the destination vector. For the synthesis of the C-terminal fragment, codon optimisation was performed due to the high complexity score. Both gBlock gene fragments were first blunt ended and ligated into the pSC-B-amp/kan plasmid using the StrataClone Blunt PCR Cloning Kit (#240207) to generate the vectors (a) pSC_Adamts12_AA1-160 and (b) pSC_Adamts12_AA611-1595-HA. The N-terminal fragment was transferred from the pSC vector into the pMIG backbone (Addgene plasmid #9044) via restriction cloning and using the restriction enzymes XhoI and EcoRI, and the plasmid pMIG-Adamts12_AA1-160 was generated. Subsequently, the N-terminus was transferred from the pSC vector into the target plasmid via EcoRI restriction cloning (“in-frame” cloning). The integration of the H465Q-E466A mutation was performed using the Q5 Site-Directed Mutagenesis Kit (NEB; #E0554) and the primers Mut-H465Q-466A-F: 5′-CACAATTGCCcaagcgCTAGGACACAG-3′ and Mut-H465-E466A-R: 5′-AAAGCCAGAGGGAGTCCC-3′. The integrated CDS (both WT- and MUT-ADAMTS12) was controlled by sequencing. Retroviral particles were produced by transient transfection in combination with the packaging plasmid pUMVC (Addgene plasmid #8449) and the pseudotyping plasmid pMD2.G (Addgene plasmid #12259; http://n2t.net/addgene:12259; RRID:Addgene_12259) using TransIT-LT (Mirus). Viral supernatants were collected 48-72 hours after transfection, clarified by centrifugation, supplemented with 10% FCS and Polybrene (Sigma-Aldrich, final concentration of 8 μg/ml) and filtered through a 0.45 μm sieve (Millipore; SLHP033RS). Cell transduction was performed by incubating the PDGFB cells with viral supernatants for 48 h. eGFP-expressing cells were purified by fluorescence-activated cell sorting.

### Western Blot:

For protein isolation, cells were lysed with RIPA buffer containing a protease inhibitor cocktail (Roche). Lysate protein concentrations were measured using the Pierce BCA Protein Assay Kit (#23225, ThermoScientific). Subsequently, equal concentration-adjusted protein lysates were denatured for 5 min at 95° C. in SDS sample loading buffer (BioRad) and loaded onto 10% SDS-Page gels. After gel electrophoresis, samples were transferred to a PVDF membrane and blots were probed with primary antibody in 5% Blotto (Thermo Fisher) (1:2000 anti-HA epitope tag (BioLegend #901533) for 2 hours, followed by incubation with secondary antibody for 1 hour after washing (horseradish peroxidase-HRP-conjugated anti-mouse antibody, Vector Laboratories) and developed using Pierce™ ECL Western Blotting Substrates A and B. The anti-tubulin monoclonal antibody and anti-GFP goat antibody (Rockland #600-101-215, 1:2000), followed by an HRP-conjugated secondary anti-mouse and anti-goat antibody, respectively (Vector Laboratories), were used as loading controls.

Migration Analyses: Cells were seeded in a Matrigel-coated 96-well plate (flat bottom, transparent, 89626, ibidi). After 24 hours of incubation with starvation medium (0.5% foetal calf serum), 50% confluent cells were stimulated with 10 ng/ml TGFβ in CO2-independent medium (18045054, Gibco). After 24 hours of stimulation, the autofluorescence of the cells was recorded every 10 minutes for 18-24 hours in a 37° C. chamber using a Nikon AIR confocal microscope. Cell segmentation was performed for each time point using ilastik's pixel classification workflow, which was then exported as so-called “prediction maps”. The prediction maps of the different points in time were then aligned and integrated for each region and the cell coordinates and average velocity were calculated using the ImageJ plugins StackReg and TrackMate. The velocities were weighted according to the length of the individual tracks. The movement of the cell was calculated with the package ggplot2 in R and displayed graphically. The diagrams show the representative results of a total of three independent experiments.

Single-Cell RNA Analysis: Single-cell RNA data collection and analysis including transcript alignment, normalisation, scaling, dimension reduction and cell annotation was performed as described (Kuppe et al., 2021). Gene expression analysis of ADAMTS12 was performed using the Seurat package in R.

### Quantification and Statistical Analysis Used Outside the Single-Cell Sequencing and Microarray Data

Data are presented as mean value ± standard deviation unless otherwise stated in the keys. The comparison of two groups was performed with an unpaired t-test. A two-way ANOVA with Tukey's multiple comparison test was used to compare multiple groups. Statistical analyses were performed using GraphPad Prism 8 (GraphPad Software Inc., San Diego, CA). A p-value of less than 0.05 was considered significant.

### Example 2: Overexpression of the ADAMTS12 Gene in Activated Fibroblasts after Kidney Damage

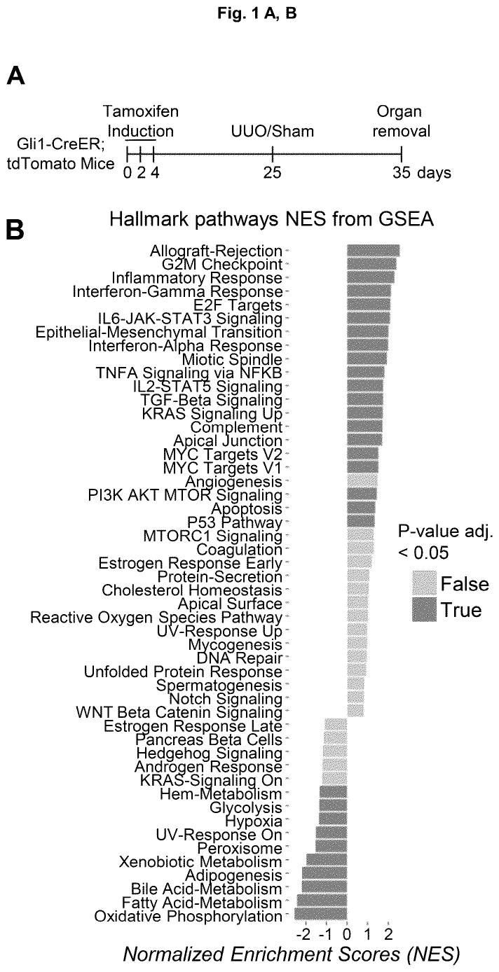

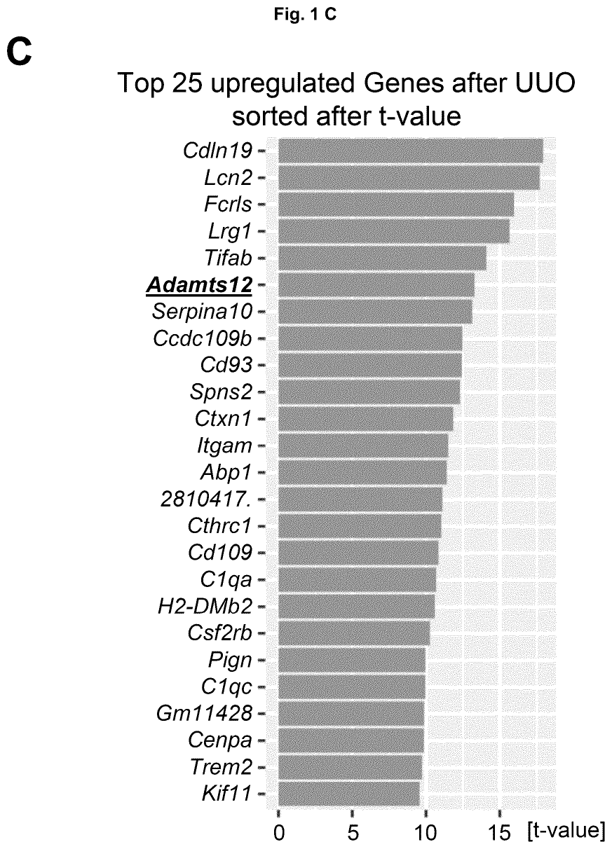

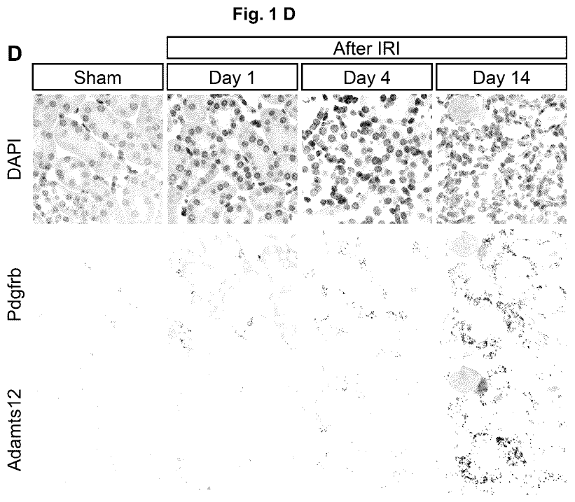

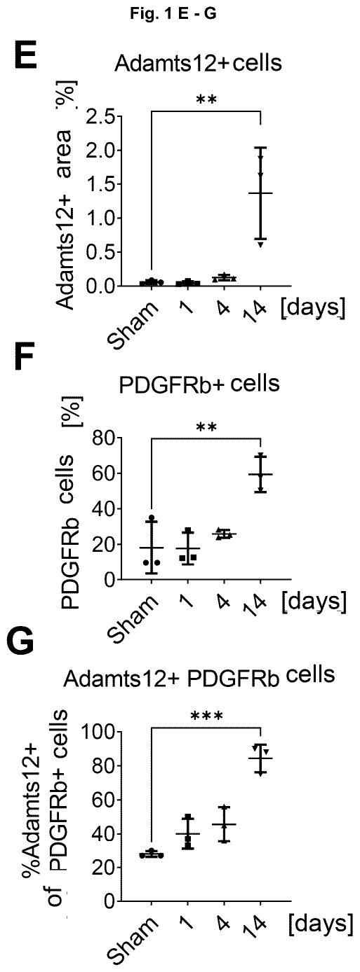

To identify new signalling pathways that could lead to activation of Gli1 fibroblasts, fibroblasts expressing the transcription factor Gli1 (Gli1 fibroblasts) were genetically labelled with the fluorophore tdTomato in Gli1-CreER^{(t2}); R26tdTomato mice by repeated administration of tamoxifen. 25 days after tamoxifen induction, either a unilateral ureteral obstruction (UUO) was performed to induce renal fibrosis or a sham surgery was performed as a control. 10 days after surgery, mice were killed, Gli1 fibroblasts were isolated from UUO or control kidneys using fluorescent activated cell sorting (FACS), and the RNA transcriptome was measured using an Affymetrix microarray assay (FIG. 1 A). Principal component analysis (PCA) was used to validate that activated Gli1 fibroblasts after UUO were clearly different from non-activated Gli1 fibroblasts from control kidneys (Sham).

Subsequently, a Gene Set Enrichment Analysis (GSEA) was performed based on the Hallmark Pathways (FIG. 1 B). The GSEA showed significantly increased normalised enrichment scores (NES) of proinflammatory pathways (inflammatory immune response, IL6-STAT3, TNFA via NFKB), myofibroblast-associated pathways (epithelial-mesenchymal transition, TGF-beta pathway) and proliferation (G2M checkpoint, mitotic spindle, E2F targets). This showed that Gli1 fibroblasts expand after UUO and differentiate into myofibroblasts. In a gene expression analysis, proinflammatory genes and extracellular matrix genes were found to be the most upregulated (FIG. 1 C). One of the most upregulated genes (top 6 ordered by T-value) in activated Gli1 fibroblasts was the ADAMTS12 gene (A Disintegrin And Metalloproteinase with ThromboSpondin motifs 12).

ADAMTS12 belongs to the family of ADAMTS metalloproteases; a function of ADAMTS12 in the pathogenesis of fibrosis was previously unknown.

To validate these new findings, RNA in situ hybridisation for ADAMTS12 and the fibroblast marker PDGFRb was performed after induction of renal fibrosis by ischaemia-reperfusion injury (IRI) (FIG. 1 D). ISH staining showed that the ADAMTS12 gene is only minimally expressed in homeostasis and is only drastically upregulated in PDGFRb-positive fibroblasts after renal damage (FIG. 1 E-G).

From these findings, the inventors were surprised to discover that ADAMTS12 is one of the most strongly upregulated genes in Gli1 fibroblasts after unilateral ureteral obstruction (UUO).

### Example 3: Reduction of Renal and Cardiac Fibrosis In Vivo by “Knockout” of ADAMTS12

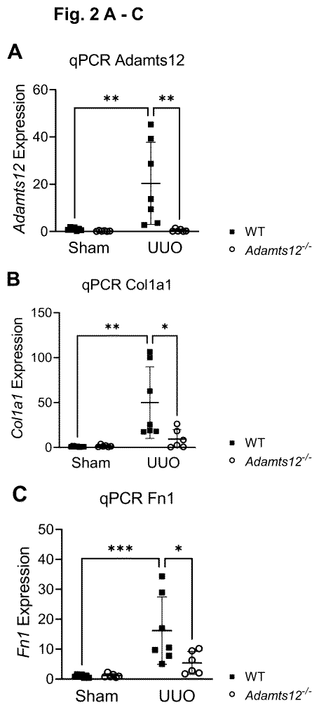

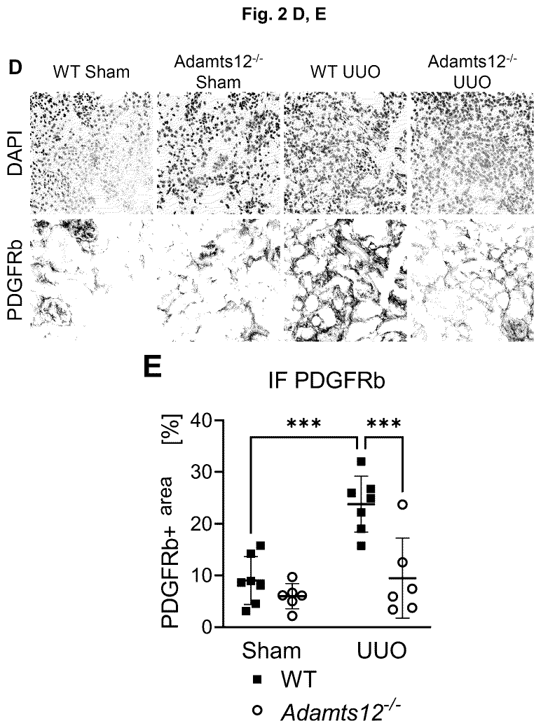

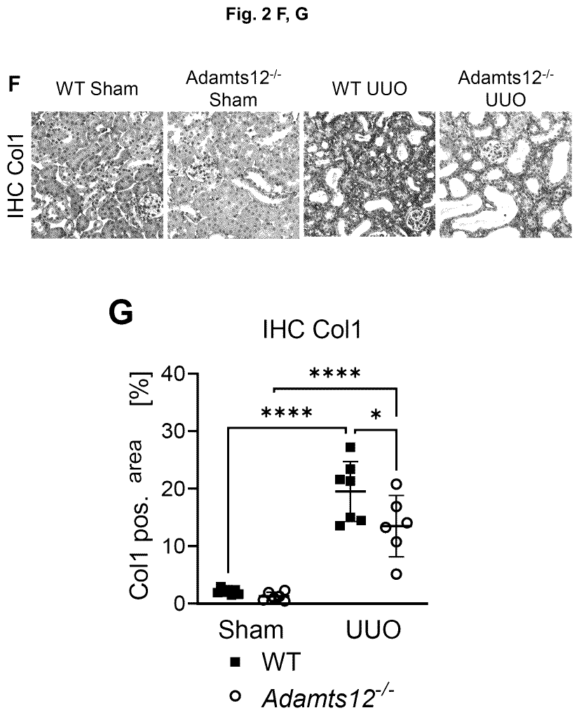

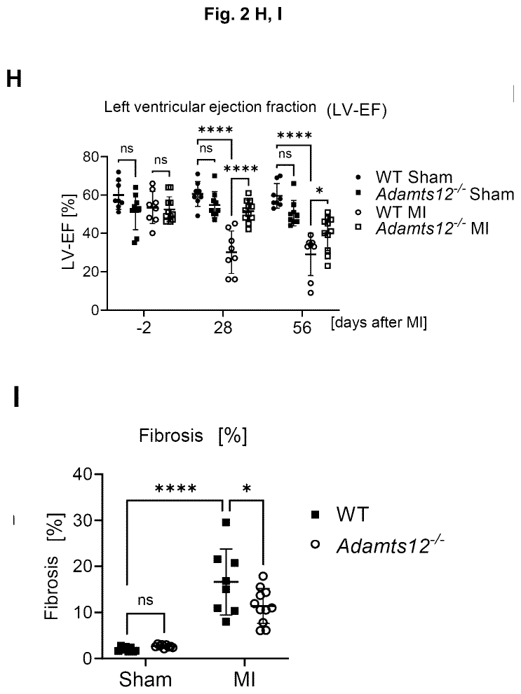

Based on the microarray data obtained, unilateral ureteral obstruction (UUO) was performed in wild-type and ADAMTS12 knockout (KO) mice (Adamts12^{-/-}). Quantitative real-time PCR (RT-qPCR) was used to determine the gene expressions for ADAMTS12 and the extracellular matrix (ECM) proteins collagen 1 (Col1a1) and fibronectin (Fn1) (FIG. 2A-C). Genetic knockout of ADAMTS12 resulted in a loss of ADAMTS12 expression at the RNA level (FIG. 2 A). In addition, ADAMTS12 knockout (KO) mice showed significantly reduced gene expression of collagen 1 and fibronectin after UUO. Immunofluorescence staining for the fibroblast marker PDGFRb and immunohistochemical staining for the ECM protein collagen 1 were performed to validate the RT-qPCR results (FIG. 2 D, 2 F). The quantification of PDGFRb showed a strongly reduced expansion of PDGFRb fibroblasts in ADAMTS12-KO mice after UUO (FIG. 2 E). Furthermore, there was a significantly reduced deposition of collagen 1 in ADAMTS12-KO mice (FIG. 2 G).

To analyse whether these results are transferable to the pathogenesis of chronic heart failure and cardiac fibrosis, myocardial infarction (MI) was induced in wild-type and ADAMTS12-KO mice by ligation of the coronary artery ramus interventricularis. Again, the genetic loss of ADAMTS12 was confirmed to lead to improved left ventricular ejection fraction (LV-EF) and reduced fibrosis after myocardial infarction (FIG. 2 H, I).

In summary, it was surprisingly found that the knockout of ADAMTS12 reduces both renal and cardiac fibrosis after organ damage, and in the case of myocardial infarction even leads to a reduced loss of function. From these results it can be concluded that ADAMTS12 plays a key role in the activation of Gli1 fibroblasts.

### Example 4: Reduction of Myofibroblast Differentiation and Migration of Human Fibroblasts In Vitro by CRISPR-CAS9-Mediated Knockout of ADAMTS12

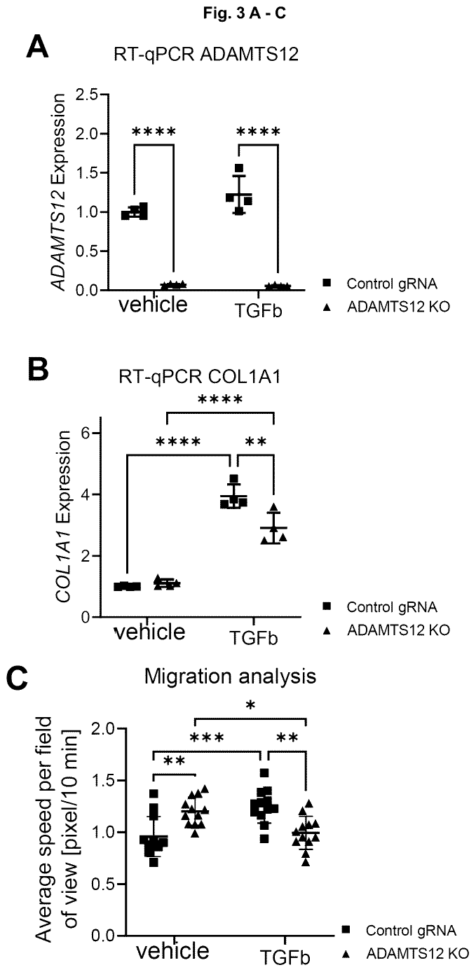

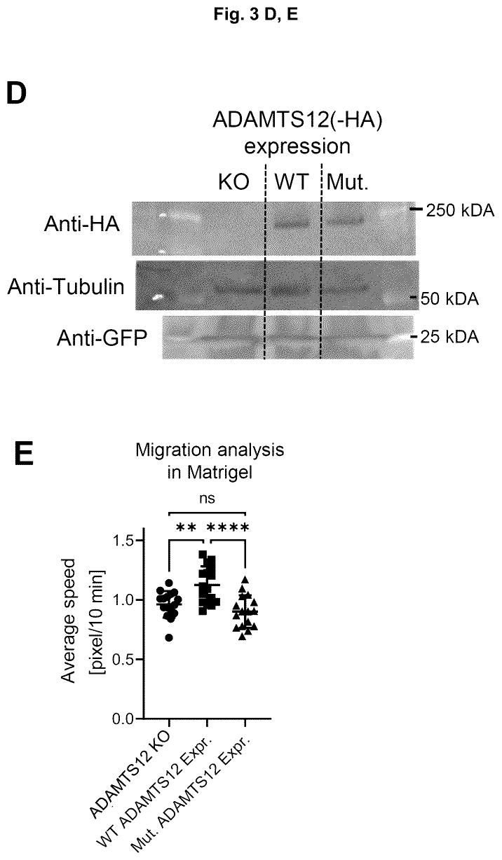

Subsequently, based on the results in vivo (Example 3, FIG. 2), the function of the metalloprotease ADAMTS12 was investigated in vitro to determine whether ADAMTS12 could be essential for the expansion and myofibroblast differentiation of fibroblasts. Knockouts (KO) of ADAMTS12 were induced in immortalised human renal PDGFRb-positive fibroblasts using CRISPR-Cas9 (FIG. 3 A). Stimulation with transforming growth factor beta (TGFb) was first used to investigate the capacity for myofibroblast differentiation in ADAMTS12-KO and WT fibroblasts. Here it was confirmed that the KO of ADAMTS12 reduces the expression of collagen 1 (COL1A1), which is a marker for myofibroblast differentiation (FIG. 3A, 3B). An important step in the activation of fibroblasts is the expansion and migration of fibroblasts from the perivascular niche into the interstitium. In a second experiment, the migration of WT and ADAMTS12-KO fibroblasts was therefore analysed using a confocal microscope. This demonstrated that the loss of ADAMTS12 significantly reduced the migration of fibroblasts after TGFb stimulation (FIG. 3 C).

To analyse whether the observed effect of ADAMTS12 is mediated by the metalloproteinase domain of ADAMTS12, catalytically active (wild type, WT) or inactive (mutant, Mut.) ADAMTS12 was expressed by retroviral transduction of an ADAMTS12-pMIG expression vector in immortalised human renal PDGFRb-positive fibroblasts in which ADAMTS12 had been “knocked out” by CRISPR-Cas9 vector as previously described (FIG. 3 D).

Overexpression of catalytically active ADAMTS12 (WT) led to increased migration of fibroblasts after activation, whereas overexpression of catalytically inactive ADAMTS12 (Mut.) did not affect migration (FIG. 3 E). These results confirm that ADAMTS12 induces fibroblast migration via the ADAMTS12 metalloproteinase domain.

KO of ADAMTS12 via CRISPR-Cas9 thus reduces myofibroblast differentiation and migration of human fibroblasts in vitro. Catalytically active ADAMTS12 induces fibroblast migration of human fibroblasts in vitro.

### Example 5: Expression of ADAMTS in Human Kidneys by Specific Fibroblast and Myofibroblast Populations

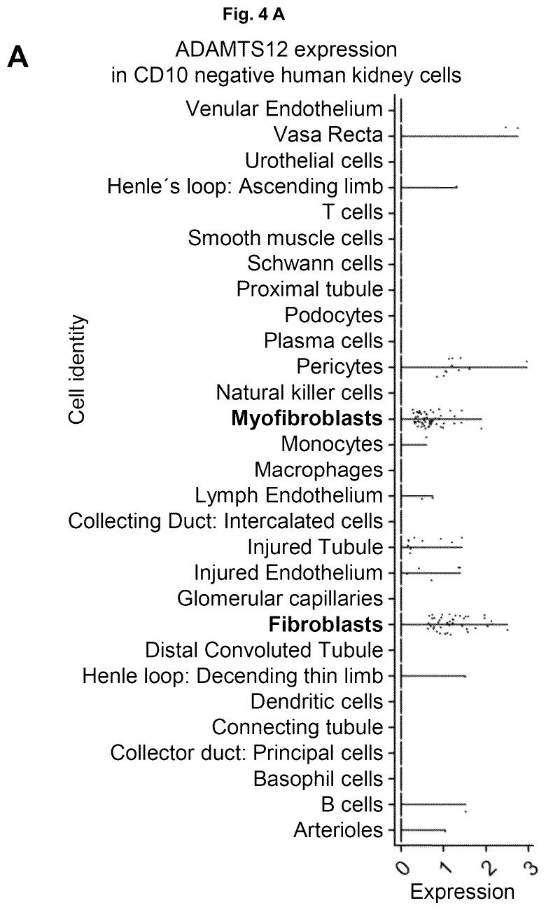

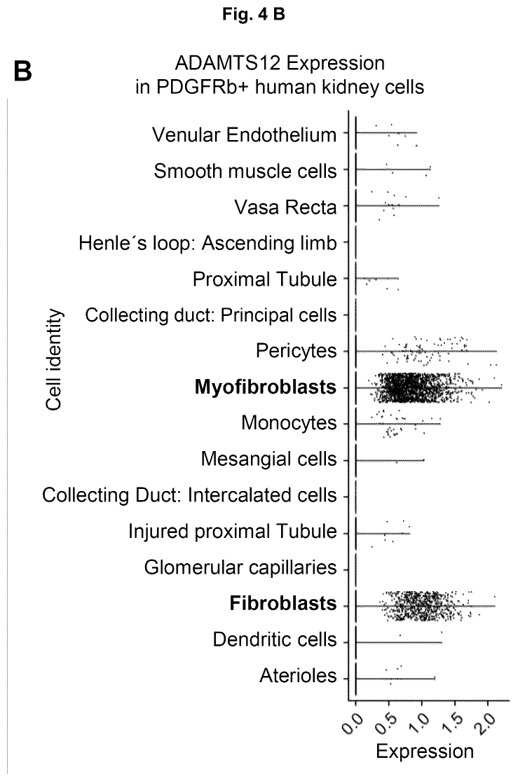

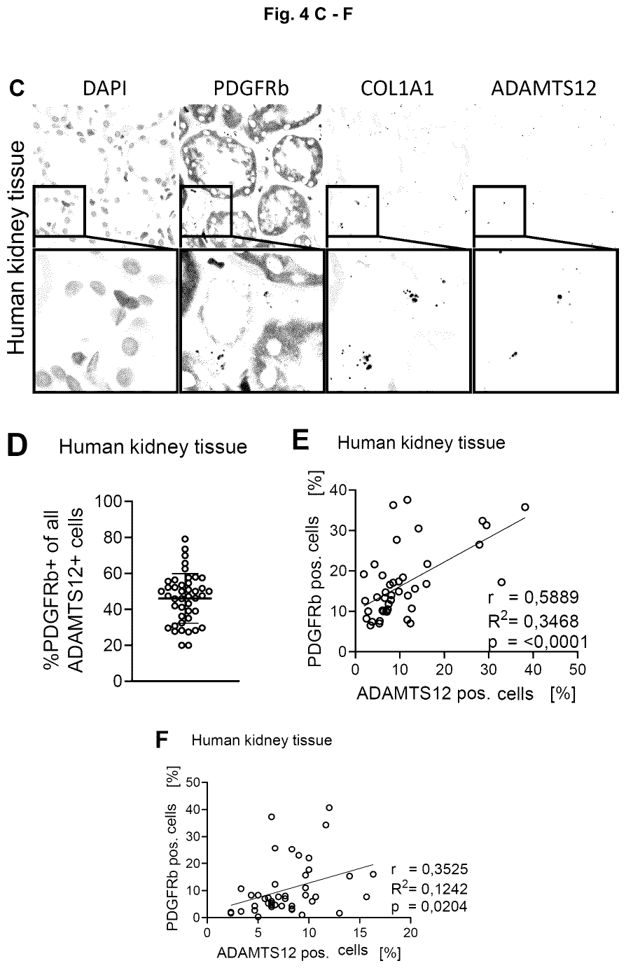

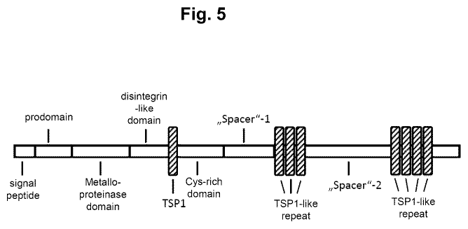

In the next step, the expression of ADAMTS12 in human kidneys was analysed. The expression of ADAMTS12 as analysed in a data set of 15 human kidneys (Kuppe et al., 2021), in which CD10-negative cells were sequenced individually (to enrich interstitial cells). This showed that ADAMTS12 is specifically expressed by fibroblasts and myofibroblasts, and to a lesser extent by pericytes (FIG. 4 A). These results were confirmed in a second data set in which PDGFRb-positive cells from eight human kidneys were single-cell sequenced (FIG. 4 B). The single cell data show that a subpopulation of fibroblasts and myofibroblasts express ADAMTS12. To validate these results, additional in situ hybridisation was performed for ADAMTS12, PDGFRb and collagen 1 (COL1A1) in 43 human kidneys (FIG. 4 C). This confirmed that ADAMTS12 is primarily produced by PDGFRb-positive fibroblasts (FIG. 4 D), and that the expression of ADAMTS12 clearly correlated with the expression of the fibroblast marker PDGFRb and the ECM protein collagen 1 (FIG. 4 E, 4 F).

### Example 6: Screening for Active Agents that Bind to and/or Inhibit the Metalloprotease ADAMTS12

Screening experiments allow the identification and validation of low molecular therapeutic compounds, peptides and/or biologics that bind to and/or inhibit the activity of the ADAMTS12 protein.

DNA-encoded substance libraries are generated and screened, as described (Kunig et al. 2018). Furthermore, phage display technologies (Takakusagi et al. 2020), cell surface display or ribosome display technologies (Galán et al. 2016) and/or combinatorial peptide libraries (Bozovicar and Bratkovic 2019) are used. For this purpose, recombinant ADAMTS12 protein or fragments thereof that can carry a “tag” for labelling, identification or purification, e.g. a His-tag or a FLAG-tag, are expressed in bacterial expression systems such as E. coli, or in insect cells or mammalian cells.

The purified ADAMTS12 protein is incubated with the substance library and isolated by immunoprecipitation. The compounds bound to the ADAMTS12 protein are identified, e.g. by Sanger sequencing of the DNA barcodes. The identified agents and compounds are then tested for their effect on the function of ADAMTS12, its protease activity, the migration of fibroblasts, the expression and secretion of matrix proteins, such as collagen 1 and fibronectin, and the development of renal fibrosis and/or cardiac fibrosis. For this purpose, experimental mouse in vivo models of renal fibrosis and cardiac fibrosis are used.

For the identification and validation of low molecular therapeutic compounds, peptides and/or biologics that exert an effect on ADAMTS12 protease activity or its expression, an in vitro human cell-based fluorochrome reporter system is established using, for example, expression of the eGFP-ADAMTS12 fusion protein or a luciferase-based reporter system to screen substance libraries in assays of 384 to 1,536 wells for the identification of compounds that reduce eGFP fluorescence or luciferase levels as readout. Expression of these human ADAMTS12 fusion reporter constructs in these cells can be performed, for example, by transfection and selection via resistance gene cassettes or by viral transduction. Human cell lines such as 293T cells, but also established human kidney fibroblast cell lines are used for these assays. In parallel to this screening, cytotoxicity assays are performed to exclude compounds that have an effect on reporter fluorescence or activity due to non-specific toxicity or induction of apoptosis.

In summary, based on the experimental data presented and using a microarray of Gli1 fibroblasts, ADAMTS12 was identified for the first time as a potential molecular target for the treatment of fibrosis In vivo, it was shown for the first time in a UUO and an MI mouse model that the knockout of ADAMTS12 strongly reduces the migration of fibroblasts and fibrosis. In vitro, knockout of ADAMTS12 using CRISPR-Cas9 reduced the migration of human renal PDGFRb-positive fibroblasts, while overexpression of catalytically active but not catalytically inactive ADAMTS12 enhanced migration. This confirms that the observed effect of ADAMTS12 is mediated by the metalloproteinase domain of ADAMTS12.

In human kidneys, ADAMTS12 was shown to be specifically produced by fibroblasts, myofibroblasts and, to a lesser extent, pericytes. Furthermore, ADAMTS12 expression correlated with the expression of the fibroblast marker PDGFRb and the fibrosis marker collagen 1.

The metalloprotease ADAMTS12 has been little studied and there are no reports of ADAMTS12 involvement in the pathogenesis of renal or cardiac fibrosis. Some studies have shown that ADAMTS12 is a negative regulator of angiogenesis (EI Hour et al., 2010), while other research groups have reported that ADAMTS12 modulates the immune response and that a knockout of ADAMTS12 leads to a prolonged proinflammatory immune response (Moncada-Pazos et al, 2018; Paulissen et al., 2012).

The metalloprotease ADAMTS12 is particularly attractive as a molecular target molecule for the treatment of fibrosis. ADAMTS12 is hardly or not at all expressed in homeostasis. After induction of renal fibrosis, ADAMTS12 is specifically upregulated in fibroblasts, pericytes and myofibroblasts. The cell-specific expression of ADAMTS12 and its low to absent expression in homeostasis suggest that inhibition of ADAMTS12 may be associated with few side effects. Furthermore, the inhibition of the metalloproteinase ADAMTS12 offers a clear starting point for the development of drugs from a biochemical point of view.

### LITERATURE

Binz, H. K., Amstutz, P., and Plückthun, A. (2005). Engineering novel binding proteins from nonimmunoglobulin domains. Nature Biotechnology Vol. 23 Nr. 10, 1257-1268.
Bozovicar, K., and Bratkovic, K. (2019). Evolving a Peptide: Library Platforms and Diversification Strategies. Int. J. Mol. Sci. 21, 215.
Berg, S. et al. (2019). ilastik: interactive machine learning for (bio)image analysis. Nature Methods 16, 1226-1232.
Cal, S., et al. (2001). Identification, characterization, and intracellular processing of ADAM-TS12, a novel human disintegrin with a complex structural organization involving multiple thrombospondin-1 repeats. J. Biol. Chem. 276, 17932-17940.
Cm, V., and F, S. (2017). A comparison between the costs of dialysis treatments in Marche Region, Italy: Macerata and Tolentino hospitals. Ann. Ist. Super. Sanita 53, 344-349.
Curaj, A., et al. (2015). Minimal invasive surgical procedure of inducing myocardial infarction in mice. J. Vis. Exp. JoVE e52197.
Djudjai, S. & Boor, P. (2019). Cellular and molecular mechanisms of kidney fibrosis. Mol. Aspects Med. 65, 16-36.
El Hour, M., Moncada-Pazos, A., Blacher, S., Masset, A., Cal, S., Berndt, S., Detilleux, J., Host, L., Obaya, A. J., Maillard, C., et al. (2010). Higher sensitivity of Adamts12-deficient mice to tumor growth and angiogenesis. Oncogene 29, 3025-3032.
Fus-Kujawa A. et al. (2021). An overview of methods and tools for transfection of eukaryotic cells in vitro. Front. Bioeng. Biotechnol. Vol. 9:701031.
Galán et al. (2016). Library-based display technologies: where do we stand? Molecular Biosystems. DOI: 10.1039/c6mb00219f.
Green M. R. and Sambrook J. (2012). Molecular Cloning. A Laboratory Manual. Fourth Edition. Cold Spring Harbor Laboratory Press.
Henderson, N. C., Rieder, F., und Wynn, T. A. (2020). Fibrosis: from mechanisms to medicines. Nature 587, 555-566.
Hosse, R. J., Rothe, A., and Power, B. E. (2006). A new generation of protein display scaffolds for molecular recognition. Protein Science 15: 14-27.
Jha, V., Garcia-Garcia, G., Iseki, K., Li, Z., Naicker, S., Plattner, B., Saran, R., Wang, A. Y.-M., and Yang, C.-W. (2013). Chronic kidney disease: global dimension and perspectives. Lancet Lond. Engl. 382, 260-272.
Kelwick, R., et al. (2015). The ADAMTS (A Disintegrin and Metalloproteinase with Thrombospondin motifs) family. Genome Biology 16:113.
Kramann, R., DiRocco, D. P., and Humphreys, B. D. (2013). Understanding the origin, activation and regulation of matrix-producing myofibroblasts for treatment of fibrotic disease. J. Pathol. 231,273-289.
Kramann, R., Schneider, R. K., DiRocco, D. P., Machado, F., Fleig, S., Bondzie, P. A., Henderson, J. M., Ebert, B. L., and Humphreys, B. D. (2015a). Perivascular Gli1+ progenitors are key contributors to injury-induced organ fibrosis. Cell Stem Cell 16, 51-66.
Kramann, R., Fleig, S. V., Schneider, R. K., Fabian, S. L., DiRocco, D. P., Maarouf, O., Wongboonsin, J., Ikeda, Y., Heckl, D., Chang, S. L., et al. (2015b). Pharmacological GL12 inhibition prevents myofibroblast cell-cycle progression and reduces kidney fibrosis. J. Clin. Invest. 125, 2935-2951.
Kunig, V. et al. (2018). DNA-encoded libraries—an efficient small molecule discovery technology for the biomedical sciences. Biol. Chem. 399(7), 691-710.
Kuppe, C. et al. (2021). Decoding myofibroblast origins in human kidney fibrosis. Nature 589, 281-286.
Lin C.-W. and Lerner R. A. (2021). Antibody libraries as tools to discover functional antibodies and receptor pleiotropism. Int. J. Mol. Sci. Vol. 22 No. 4123. https://doi.org/10.3390/ijms22084123.
Lin, E. A., and Liu, C. (2009). The emerging roles of ADAMTS-7 and ADAMTS-12 matrix metalloproteinases. Open Access Rheumatology Research and Reviews. Vol. 1, 121-131.
Martin A. et al. (2020). Navigating the DNA encoded libraries chemical space. Communications Chemistry Vol. 3 No. 127. https://doi.org/10.1038/s42004-020-00374-1.
Mohamedi, Y., et al. (2021). ADAMTS-12: Functions and Challenges for a Complex Metalloprotease. Frontiers in Molecular Biosciences. Vol. 8, Article 686763.
Moncada-Pazos, A., Obaya, A. J., Lamazares, M., Heljasvaara, R., Suarez, M. F., Colado, E., Noal, A., Cal, S., and López-Otin, C. (2018). ADAMTS-12 metalloprotease is necessary for normal inflammatory response. J. Biol. Chem. 293, 11648.
Naylor, K. L., Kim, S. J., McArthur, E., Garg, A. X., McCallum, M. K., and Knoll, G. A. (2019). Mortality in Incident Maintenance Dialysis Patients Versus Incident Solid Organ Cancer Patients: A Population-Based Cohort. Am. J. Kidney Dis. Off J. Natl. Kidney Found. 73, 765-776.
Paulissen, G., El Hour, M., Rocks, N., Gueders, M. M., Bureau, F., Foidart, J.-M., López-Otin, C., Noel, A., and Cataldo, D. D. (2012). Control of allergen-induced inflammation and hyperresponsiveness by the metalloproteinase ADAMTS-12. J. Immunol. Baltim. Md 1950 189, 4135-4143.
Saldivar-Gonzalez F. I. et al. (2020). Chemoinformatics-based enumeration of chemical libraries: a tutorial. Journal of Cheminformatics Vol. 12 No. 64. https://doi.org/10.1186/s13321-020-00466-z.
Schwaar T. et al. (2019). Efficient screening of combinatorial peptide libraries by spatially ordered beads immobilized on conventional glass slides. High-Throughput Vol. 8 No. 11. doi:10.3390/ht8020011.
Takakusagi et al. (2020) Phage display technology for target determination of small-molecule therapeutics:an update. Expert Opinion on Drug Discovery. Vol. 15, No. 10, 1199-1211.
Valldorf B. et al., (2022). Antibody display technologies: selecting the cream of the crop. Biol. Chem. Vol. 403 No. 5-6, p. 455-477.
Volochnyuk D. M. et al. (2019). Evolution of commercially available compounds for HTS. Drug Discovery Today Vol. 24 No. 2, p. 390-402.
Wassermann A. M. et al. (2014). Composition and applications of focus libraries to pheno-typic assays. Frontiers in Pharmacology Vol. 5 No. 164. doi: 10.3389/fphar.2014.00164.
Wei, J., Richbourgh, B., Jia, T., and Liu, C. (2014). ADAMTS-12: a multifaced metalloproteinase in arthritis and inflammation. Mediators Inflamm. 2014, 649718.
Witten, A., et al. (2020). ADAMTS12, a new candidate gene for pediatric stroke. PLoS ONE 15(8): e0237928.

1. A method for reducing the expression and/or secretion of extracellular matrix (ECM) proteins by a given cell, and/or for inhibiting the migration of fibroblasts, wherein the method comprises at least one step selected from the group consisting of
(i) inhibition or reduction of ADAMTS12 gene expression in the cell,
(ii) inhibition or reduction of ADAMTS12 activity,
(iii) inhibition or reduction of ADAMTS12 protease activity, and/or
(iv) promotion of the degradation of the ADAMTS12 protein.

2. The method according to claim 1, wherein the inhibition or reduction of ADAMTS12 gene expression comprises ADAMTS12 gene knock-down, knock-out, conditional gene knock-out, gene modification, RNA interference, siRNA and/or antisense RNA.

3. The method according to claim 1, wherein the inhibition or reduction of ADAMTS12 activity comprises the use of an active agent that binds to the ADAMTS12 protein (A Disintegrin And Metalloproteinase with ThromboSpondin motifs 12 protein).

4. The method according to claim 1, wherein said cell is a renal cell or a cardiac cell, preferably a renal fibroblast cell or a cardiac fibroblast cell, a renal myofibroblast cell or a cardiac myofibroblast cell, or a renal pericyte or cardiac pericyte; most preferably a renal fibroblast cell or a cardiac fibroblast cell.

5. A method for identifying an active agent that binds to the ADAMTS12 protein (A Disintegrin And Metalloproteinase with ThromboSpondin motifs 12 protein) or a fragment thereof, and/or inhibits or reduces the activity of the ADAMTS12 protein, or a fragment thereof, comprising at least the following steps:
(i) providing the ADAMTS12 protein or a fragment thereof,
(ii) adding at least one active agent to be analysed for binding to the ADAMTS12 protein or a fragment thereof, and
(iii) identifying the at least one active agent that has bound to the ADAMTS12 protein or a fragment thereof.

6. (canceled)

7. The method according to claim 5, wherein the active agent is an ADAMTS12 inhibitor.

8. The method according to claim 5, wherein the active agent is a member of a library of compounds.

9. The method according to claim 5, wherein the active agent is selected from the group consisting of a low molecular compound, a peptide and a biologic.

10. (canceled)

11. The method according to claim 5, wherein the ADAMTS12 protein is bound to a solid phase or is present in solution.

12.-14. (canceled)

15. An active agent which binds to the ADAMTS12 protein (A Disintegrin And Metalloproteinase with ThromboSpondin motifs 12 protein) or a fragment thereof, and/or inhibits or reduces the activity of the ADAMTS12 protein, or a fragment thereof, and/or promotes the degradation of the ADAMTS12 protein.

16. The active agent according to claim 15, wherein the active agent is a low molecular compound (smol), a peptide or a biologic, wherein the biologic is an antibody or a fragment thereof, a derivative thereof, an antibody-like protein, or an aptamer.

17. (canceled)

18. (canceled)

19. The active agent according to claim 16, or antibody, antigen-binding fragment or antigen-binding derivative thereof, or antibody-like protein, for use in the treatment of chronic kidney disease and/or heart disease.

20. An active agent or antibody, antigen-binding fragment or antigen-binding derivative thereof, or antibody-like protein, for use according to claim 19, wherein the chronic kidney disease is progressive chronic renal insufficiency and/or renal fibrosis, and/or wherein the heart disease is heart failure and/or cardiac fibrosis.

21.-23. (canceled)

24. A method for treating or preventing chronic kidney disease and/or heart disease, wherein the method comprises administering to a human or animal subject a therapeutically effective dose of an active agent that binds to and/or inhibits the ADAMTS12 protein.

25.-30. (canceled)
